(19) United States
(12) Patent Application Publication
(10) Pub. No.: US 2010/0029941 A1
(43) Pub. Date: Feb. 4, 2010

(54) PREPARATION OF (R)-3-AMINOPIPERIDINE DIHYDROCHLORIDE

(75) Inventors: Michael B. Wallace, San Diego, CA (US); Jeremy Cody, Clifton Springs, NY (US); Richard S. Fornicola, Liverpool, NY (US); Silvina Garcia-Rubio, Manlius, NY (US); Philip B. Kisanga, Cicero, NY (US); Maxwell D. Reeve, Guilford, CT (US); Chandra Wilson, Syracuse, NY (US)

Correspondence Address: TAKEDA SAN DIEGO, INC., 10410 SCIENCE CENTER DRIVE, SAN DIEGO, CA, 92121, US

(73) Assignee: TAKEDA PHARMACEUTICAL COMPANY LIMITED, Osaka (JP)

(21) Appl. No.: 12/294,018

(22) PCT Filed: Mar. 26, 2007
(86) PCT No.: PCT/US07/64958

§ 371 Date: Jun. 25, 2009

Related U.S. Application Data
(60) Provisional application No. 60/787,058, filed on Mar. 28, 2006.

(51) Int. Cl. C07D 401/14

(52) U.S. Cl. 544/310

(57) ABSTRACT

Described herein are methods for making chiral derivatives of 3-aminopiperidine, including the production of such derivatives in quantities exceeding 1 kilogram. The chiral 3-aminopiperidine derivatives include (R)-3-aminopiperidine derivatives which may be used to synthesize inhibitors of Dipeptidyl Peptidase IV. (R)-3-Aminopiperidine dihydrochloride is prepared by reducing (R)-3-aminopiperidin-2-one hydrochloride with lithium aluminum hydride. The preparation of (R)-3-aminopiperidin-2-one hydrochloride starting from (R)-methyl-2,5-diaminopentanoate dihydrochloride or (R)-2,5-diaminopentanoic acid hydrochloride is also described.

### FIELD OF THE INVENTION

Described herein are methods for synthesizing chiral piperidine compounds as well as the chiral piperidines produced by such synthetic methods. Also described herein are methods for synthesizing dipeptidyl peptidase inhibitors from the chiral piperidines.

### BACKGROUND OF THE INVENTION

Drugs frequently possess one or more chiral centers. In medicinal chemistry, it is recognized that different enantiomers of a compound will possess different biological activities, safety profiles, pharmacokinetic properties, and pharmacodynamic properties. It is therefore desirable to be able to administer only the enantiomer of a given compound that has the most advantageous drug properties. It is thus desirable to have synthetic routes that allow a given drug to be prepared with high enantiomeric purity. Additionally, the synthetic route needs to be scalable, reproducible, and cost-effective.

### SUMMARY OF THE INVENTION

Described herein are methods for synthesizing and purifying chiral piperidine derivatives, including the R- and S-forms of chiral piperidine derivatives. Also presented herein are methods for synthesizing and purifying chiral compounds that can be used to produce chiral piperidine derivatives. In one embodiment, the chiral piperidine derivatives and their synthetic intermediates are produced in quantities exceeding 1 kilogram. In further or alternative embodiments, the chiral piperidine derivatives and their synthetic intermediates are produced in high yield and high enantiomeric purity. In further or alternative embodiments, the chiral piperidine derivatives are used to produce inhibitors of Dipeptidyl Peptidase IV. In further or alternative embodiments, the chiral piperidine derivative is 3-aminopiperidine dihydrochloride. Although certain embodiments presented herein use, as an example, one enantiomer of a compound, the methods, compositions and techniques presented herein may also be used with the corresponding opposite enantiomer of the compound.

In one aspect, the present invention relates to methods for synthesizing (R)-3-aminopiperidine dihydrochloride by: (i) mixing between about 1.0 and about 2.5 equivalents of lithium aluminum hydride in a first solvent which contains tetrahydrofuran with about 1 equivalent of (R)-3-aminopiperidin-2-one hydrochloride in a second solvent which contains tetrahydrofuran at a temperature between about 10° C. and about 45° C.; (ii) heating the reaction mixture at a temperature between about 45° C. and about 70° C. for a time sufficient to form (R)-3-aminopiperidine, and (iii) admixing the (R)-3-aminopiperidine with hydrochloric acid under conditions sufficient to form the (R)-3-aminopiperidine dihydrochloride.

In another embodiment, the first and second solvents are tetrahydrofuran. In a further or alternative embodiment, between about 1.5 and about 2.0 equivalents of lithium aluminum hydride are added in step (i), while in a still further or alternative embodiment about 1.6 equivalents of lithium aluminum hydride are added in step (i). In another embodiment, the amount of (R)-3-aminopiperidin-2-one hydrochloride used in step (i) is at least about a kilogram. In a further or alternative embodiment, the temperature of step (i) is between about 25° C. and 35° C., while in a further or alternative embodiment, the temperature of step (ii) is between about 55° C. and about 65° C. In another embodiment, the (R)-3-aminopiperidine dihydrochloride is isolated by a method comprising filtration.

In a further or alternative embodiment, the (R)-3-aminopiperidin-2-one hydrochloride is synthesized by a method which involves: (i) mixing between about 1.5 and about 3 equivalents of sodium methoxide in a first solvent which contains methanol with about 1 equivalent of (R)-methyl 2,5-diaminopentanoate dihydrochloride in a second solvent which contains methanol at a temperature between about −10° C. and about 0° C. for a time sufficient to form (R)-3-aminopiperidin-2-one, and (ii) mixing between about 1.0 and about 1.5 equivalents of hydrochloric acid in a third solvent which contains methanol with the (R)-3-aminopiperidin-2-one from step (i), at a temperature between about 0° C. and about 20° C. for a time sufficient to form (R)-3-aminopiperidin-2-one hydrochloride.

In a further or alternative embodiment, at least one of the first and second solvents in step (i) is methanol, while in a still further or alternative embodiment of this method, at least one of the third and fourth solvents in step (ii) contains methyl tert-butyl ether and methanol. In an even further or alternative embodiment, about 2.6 equivalents of sodium methoxide are added in step (i). In a further or alternative embodiment, the temperature of step (i) is between about −10° C. and about −5° C., while in a further or alternative embodiment, the temperature of step (ii) is between about 5° C. and about 15° C. In a still further or alternative embodiment, the (R)-3-aminopiperidin-2-one hydrochloride is isolated by a method comprising filtration.

In a further or alternative embodiment of the above method for the synthesis of (R)-3-aminopiperidin-2-one hydrochloride, the (R)-methyl 2,5-diaminopentanoate dihydrochloride is synthesized by a method which involves: (i) mixing between about 1.5 and about 2.5 equivalents of acetyl chloride in a first solvent containing methanol with about 1 equivalent of (R)-2,5-diaminopentanoic acid hydrochloride in a second solvent containing methanol at a temperature between about 0° C. and about 15° C., and (ii) heating the reaction mixture at a temperature between about 45° C. and about 65° C. for a time sufficient to form (R)-methyl 2,5-diaminopentanoate dihydrochloride. In an embodiment of this method, at least one of the first and second solvents is methanol. In a further or alternative embodiment of this method, about 2.0 equivalents of acetyl chloride are added in step (i). In a still further or alternative embodiment of this method, at least about 1 kilogram of (R)-2,5-diaminopentanoic acid hydrochloride is added in step (i). In a further or alternative embodiment of this method, the temperature of step (i) is between about 5° C. and about 10° C., while in an even further or alternative embodiment of this method, the temperature of step (ii) is between about 50° C. and about 60° C.

Another aspect are methods for the synthesis of (R)-3-aminopiperidine dihydrochloride which involves admixing (R)-3-aminopiperidine with concentrated hydrochloric acid, and wherein the (R)-3-aminopiperidine is synthesized by a method involving: (i) adding between about 1.6 equivalents of lithium aluminum hydride in tetrahydrofuran to about 1 equivalent of (R)-3-aminopiperidin-2-one hydrochloride in tetrahydrofuran at a temperature about 35° C., and (ii) heating the reaction mixture at a temperature between about 58° C. and about 60° C. In an embodiment of this aspect, the amount of (R)-3-aminopiperidin-2-one hydrochloride used step (i) is at least about 4000 grams. In a further or alternative embodiment, the (R)-3-aminopiperidine dihydrochloride is obtained by filtering the admixture. In another embodiment of this aspect, the (R)-3-aminopiperidin-2-one hydrochloride is synthesized by a method which involves: (i) adding between about 2.6 equivalents of sodium methoxide in methanol to about 1 equivalent of (R)-methyl 2,5-diaminopentanoate dihydrochloride in methanol at a temperature between about −10° C. and about −5° C., for a time sufficient to form (R)-3-aminopiperidin-2-one, and (ii) adding between about 1.0 and about 1.5 equivalents of acetyl chloride in a solvent comprising methyl tert-butyl ether and methanol to about 1 equivalent of (R)-3-aminopiperidin-2-one from step (i) in a solvent comprising methanol at a temperature between about 5° C. and about 15° C. In a further or alternative embodiment, the (R)-3-aminopiperidin-2-one is obtained by filtering the reaction mixture. In an embodiment of the method for the synthesis of (R)-3-aminopiperidin-2-one hydrochloride, the (R)-methyl 2,5-diaminopentanoate dihydrochloride is synthesized by a method involving: (i) adding between about 2.0 equivalents of acetyl chloride in methanol to about 1 equivalent of (R)-2,5-diaminopentanoic acid hydrochloride in methanol at a temperature between about 0° C. and about 10° C., and (ii) heating the reaction mixture at a temperature between about 50° C. and about 60° C. In an embodiment of this method, the solvent is methanol, while in further or alternative embodiment of this method, the amount of (R)-2,5-diaminopentanoic acid hydrochloride used in step (i) is at least about 1000 grams.

In another aspect are methods for the synthesis of (R)-3-aminopiperidine dihydrochloride involving admixing (R)-3-aminopiperidine with concentrated hydrochloric acid; wherein (a) the (R)-3-aminopiperidine is synthesized by a method involving: (ia) adding between about 1.0 and about 2.5 equivalents of lithium aluminum hydride in a solvent containing tetrahydrofuran to about 1 equivalent of (R)-3-aminopiperidin-2-one hydrochloride in a solvent containing tetrahydrofuran at a temperature between about 10° C. and about 45° C., and (iia) heating the reaction mixture at a temperature between about 45° C. and about 70° C.; (b) wherein the (R)-3-aminopiperidin-2-one hydrochloride from step (i) is synthesized by a method involving: (ib) adding between about 1.5 and about 3 equivalents of sodium methoxide in a solvent containing methanol to about 1 equivalent of (R)-methyl 2,5-diaminopentanoate dihydrochloride in a solvent containing methanol at a temperature between about −10° C. and about 0° C., whereby synthesizing (R)-3-aminopiperidin-2-one, and (iib) adding between about 1.0 and about 1.5 equivalents of hydrochloric acid in a solvent containing methanol to about 1 equivalent of (R)-3-aminopiperidin-2-one from step (i) in a solvent containing methanol at a temperature between about 0° C. and about 20° C.; and (c) wherein the (R)-methyl 2,5-diaminopentanoate dihydrochloride of step (ib) is synthesized by a method involving (ic) adding between about 1.5 and about 2.5 equivalents of acetyl chloride in a solvent containing methanol to about 1 equivalent of (R)-2,5-diaminopentanoic acid hydrochloride in a solvent containing methanol at a temperature between about 0° C. and about 15° C., and (iic) heating the reaction mixture at a temperature between about 45° C. and about 65° C.

In an embodiment of this aspect, the solvent of step (ia) is tetrahydrofuran, while in a further or alternative embodiment, about 1.6 equivalents of lithium aluminum hydride are added in step (ia). In an even further or alternative embodiment, the amount of (R)-3-aminopiperidin-2-one hydrochloride used in step (ia) is at least about 1000 grams, while in a still further or alternative embodiment, the temperature of step (ia) is about 35° C. In another embodiment of this aspect, the temperature of step (iia) is between about 58° C. and about 60° C., while in a further or alternative embodiment, the (R)-3-aminopiperidine dihydrochloride is obtained by filtering the admixture. In a further or alternative embodiment, the solvent in step (ib) is methanol, while in a further or alternative embodiment, the solvent in step (iib) contains methyl tert-butyl ether and methanol. In an even further or alternative embodiment, about 2.6 equivalents of sodium methoxide are added in step (ib), while in a further or alternative embodiment, the temperature of step (ib) is between about −10° C. and about −5° C. In a still further or alternative embodiment, the temperature of step (iib) is between about 5° C. and about 15° C. In a further or alternative embodiment, the (R)-3-aminopiperidin-2-one is obtained by filtering the reaction mixture. In another embodiment of this aspect, the solvent in step (1c) is methanol, while in a further or alternative embodiment, about 2 equivalents of acetyl chloride are added in step (ic). In an even further or alternative embodiment, the amount of (R)-2,5-diaminopentanoic acid hydrochloride in step (ic) is at least about 1000 grams. In a further or alternative embodiment, the temperature of step (ic) is between about 5° C. and about 10° C., while in a further or alternative embodiment, the temperature of step (iic) is between about 50° C. and about 60° C.

Another aspect are methods for the synthesis of (R)-3-aminopiperidine dihydrochloride comprising admixing (R)-3-aminopiperidine with concentrated hydrochloric acid, wherein the (R)-3-aminopiperidine is synthesized by a method involving: (i) adding at least 14 kg of lithium aluminum hydride in a solvent containing tetrahydrofuran to at least 4 kg of (R)-3-aminopiperidin-2-one hydrochloride in a solvent containing tetrahydrofuran at a temperature between about 10° C. and about 45° C., and (ii) heating the reaction mixture at a temperature between about 45° C. and about 70° C. In an embodiment of this aspect the solvent is tetrahydrofuran, while in a further or alternative embodiment, the temperature of step (i) is about 35° C. In a further or alternative embodiment, the temperature of step (ii) is between about 55° C. and about 65° C., while in a further or alternative embodiment, the (R)-3-aminopiperidine dihydrochloride is obtained by filtering the admixture.

In an embodiment of this aspect the (R)-3-aminopiperidin-2-one hydrochloride is synthesized by a method involving: (i) adding at least 14 kg of sodium methoxide in a solvent containing methanol to at least 5 kg of (R)-methyl 2,5-diaminopentanoate dihydrochloride in a solvent containing methanol at a temperature between about −10° C. and about 0° C., whereby synthesizing (R)-3-aminopiperidin-2-one, and (ii) adding between about 1.0 and 1.5 equivalents of hydrochloric acid in a solvent containing methanol to about 1 equivalent of (R)-3-aminopiperidin-2-one from step (i) in a solvent containing methanol at a temperature between about 0° C. and about 20° C. In a further embodiment of the method for the synthesis of (R)-3-aminopiperidin-2-one hydrochloride, the solvent in step (i) is methanol. In an even further or alternative embodiment, the solvent in step (ii) comprises methyl tert-butyl ether and methanol, while an even further or alternative embodiment, the temperature of step (i) is between about −10° C. and about −5° C. In a still further or alternative embodiment, the temperature of step (ii) is between about 5° C. and about 15° C., while in an even further or alternative embodiment, the (R)-3-aminopiperidin-2-one hydrochloride is obtained by filtering the reaction mixture.

In an embodiment of the above synthesis of the (R)-3-aminopiperidin-2-one hydrochloride, the (R)-methyl 2,5-diaminopentanoate dihydrochloride is synthesized by an esterification method involving: (i) adding between about 1.5 and about 2.5 equivalents of acetyl chloride in a solvent containing methanol to at least about 5 kg of (R)-2,5-diaminopentanoic acid hydrochloride in a solvent containing methanol at a temperature between about 0° C. and about 15° C., and (ii) heating the reaction mixture at a temperature between about 45° C. and about 65° C. In an embodiment of this method, the solvent is methanol, while in a further or alternative embodiment, about 2 equivalents of acetyl chloride are added in step (i). In an even further or alternative embodiment of this method, the temperature of step (i) is between about 5° C. and about 10° C., while in a still further or alternative embodiment, the temperature of step (ii) is between about 50° C. and about 60° C.

Another aspect are methods for synthesizing (R)-3-aminopiperidine dihydrochloride involving admixing (R)-3-aminopiperidine with concentrated hydrochloric acid, wherein the (R)-3-aminopiperidine is synthesized by a method involving: (i) adding between about 1.0 and about 2.5 equivalents of lithium aluminum hydride in a solvent containing tetrahydrofuran to about 1 equivalent of (R)-3-aminopiperidin-2-one hydrochloride in a solvent containing tetrahydrofuran at a temperature greater than 5° C., and (ii) heating the reaction mixture at a temperature greater than 35° C. In an embodiment of this aspect, the solvents are tetrahydrofuran, while in a further or alternative embodiment, about 1.6 equivalents of lithium aluminum hydride are added in step (i). In a further or alternative embodiment, the amount of (R)-3-aminopiperidin-2-one hydrochloride used in step (i) is at least about 1 kilogram, while in an even further or alternative embodiment, the (R)-3-aminopiperidine is not isolated by vacuum distillation.

In another embodiment of this aspect, the (R)-3-aminopiperidin-2-one hydrochloride is synthesized by a method involving: (i) adding between about 1.5 and about 3 equivalents of a base in a solvent containing methanol to about 1 equivalent of (R)-methyl 2,5-diaminopentanoate dihydrochloride in a solvent containing methanol at a temperature between about -10° C. and about 0° C. for a time sufficient to form (R)-3-aminopiperidin-2-one, wherein the base is not an anion-exchange resin, and (ii) adding between about 1.0 and about 1.5 equivalents of hydrochloric acid in a solvent containing methanol to the (R)-3-aminopiperidin-2-one from step (i), in a solvent containing methanol at a temperature between about 0° C. and about 20° C. for a time sufficient to form (R)-3-aminopiperidin-2-one hydrochloride. In an embodiment of this method, the solvent in step (i) is methanol, while in a further or alternative embodiment, the solvent in step (ii) contains methyl tert-butyl ether and methanol. In a further or alternative embodiment of this method, the temperature of step (i) is between about −10° C. and about −5° C., while in a even further or alternative embodiment, the temperature of step (ii) is between about 5° C. and about 15° C. In a still further or alternative embodiment of this method, the (R)-3-aminopiperidin-2-one hydrochloride is isolated by a method comprising filtration.

Another aspect are processes of making (R)-3-aminopiperidine dihydrochloride, wherein the (R)-3-aminopiperidine dihydrochloride is formed by a reaction of (R)-3-aminopiperidine with hydrochloric acid, and wherein the (R)-3-aminopiperidine is synthesized by a process involving: (i) synthesis of (R)-methyl 2,5-diaminopentanoate dihydrochloride by an esterification reaction of (R)-2,5-diaminopentanoic acid hydrochloride with methanol and acetyl chloride; (ii) synthesis of (R)-3-aminopiperidin-2-one hydrochloride by a cyclization reaction of(R)-methyl 2,5-diaminopentanoate dihydrochloride with a metal alkoxide in an alcohol, and a reaction with hydrochloric acid in methyl tert-butyl ether; and (iii) reduction of (R)-3-aminopiperidin-2-one hydrochloride with lithium aluminum in THF at a temperature at least about 35° C. An embodiment of this aspect is the (R)-3-aminopiperidine dihydrochloride made using this process.

In another aspect are methods for synthesizing DPP-IV inhibitors that involve reaction of (R)-3-aminopiperidine dihydrochloride, produced by the above process, with a pyrimidine derivative. In an aspect of this method the pyrimidine derivative comprises a compound having the structure corresponding to the following formula,

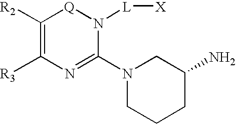

wherein:
Q is selected from the group consisting of CO, SO, SO₂, and C═NR₄;
Z′ is a leaving group;
R₂ and R₃ are each independently selected from the group consisting of hydrogen, halo, perhalo(C₁₋₁₀)alkyl, amino, cyano, nitro, thio, (C₁₋₁₀)alkyl, alkene, alkyne, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl group, imine group, sulfonyl group and sulfinyl group, each substituted or unsubstituted;
R₄ is selected from the group consisting of hydrogen, (C₁₋₁₀)alkyl, cycloalkyl, heterocycloalkyl, aryl, heteroaryl, arylalkyl, heteroarylalkyl, bicycloaryl, heterobicycloaryl, each substituted or unsubstituted;
L is a linker providing 0-6 atom separation between X and the ring to which L is attached; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

In a further or alternative embodiment, the Z′ is a halogen, also referred to as halo. In a still further or alternative embodiment, the reaction of the (R)-3-aminopiperidine dihydrochloride, produced by the above process, with the pyrimidine derivative provides a product comprising a compound having the structure according to the following formula,

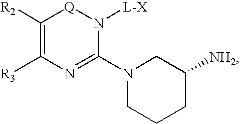

wherein:
Q is selected from the group consisting of CO, SO, SO₂, and C═NR₄;
R₂ and R₃ are each independently selected from the group consisting of hydrogen, halo, perhalo(C₁₋₁₀)alkyl, amino, cyano, nitro, thio, (C₁₋₁₀)alkyl, alkene, alkyne, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl group, imine group, sulfonyl group and sulfinyl group, each substituted or unsubstituted;
R₄ is selected from the group consisting of hydrogen, (C₁₋₁₀)alkyl, cycloalkyl, heterocycloalkyl, aryl, heteroaryl, arylalkyl, heteroarylalkyl, bicycloaryl, heterobicycloaryl, each substituted or unsubstituted;
L is a linker providing 0-6 atom separation between X and the ring to which L is attached; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

In even further or alternative embodiments, the reaction of the (R)-3-aminopiperidine dihydrochloride, produced by the above process, with the pyrimidine derivative provides a product comprising a compound selected from the group consisting of

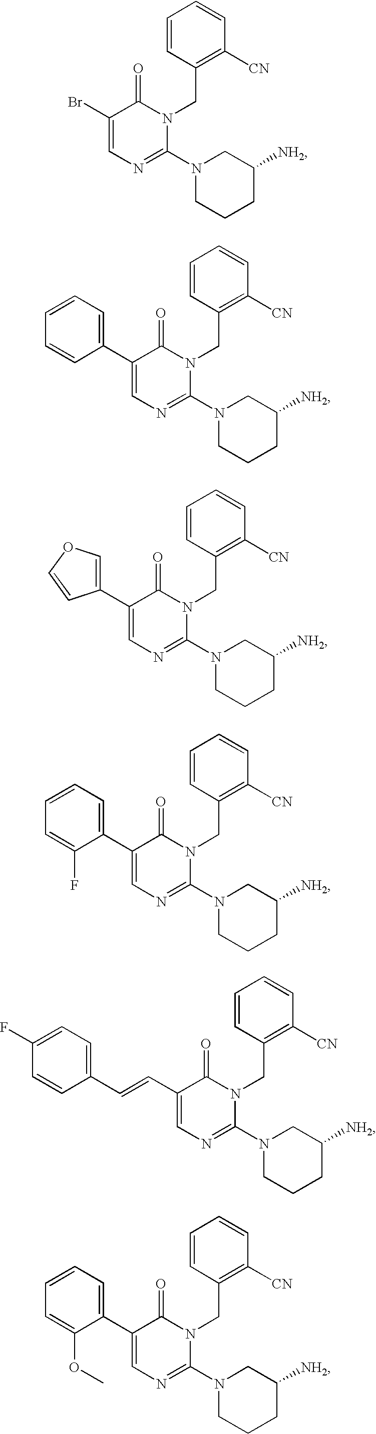

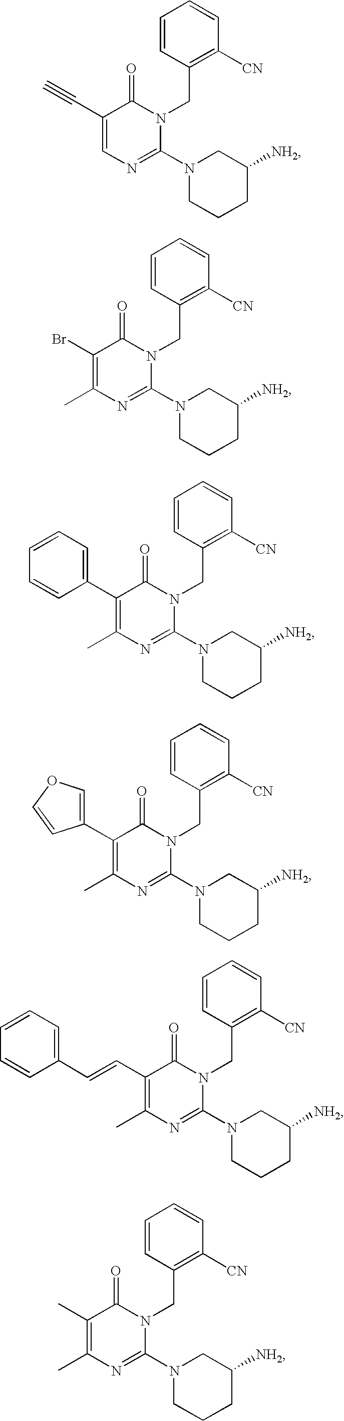

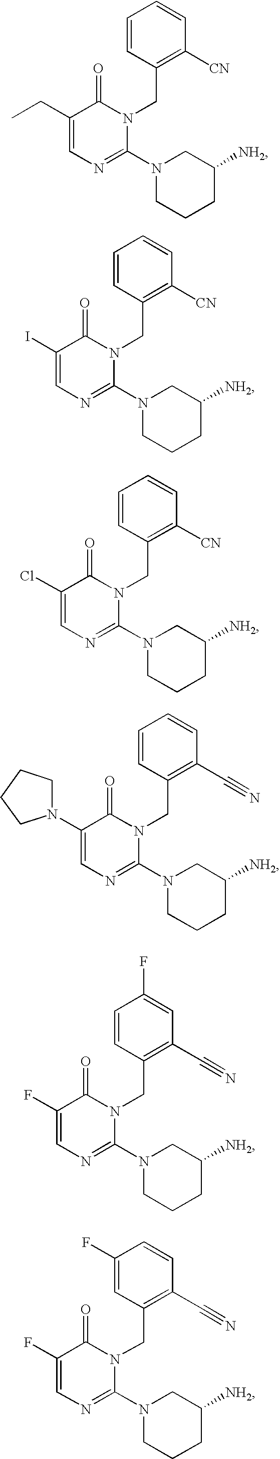

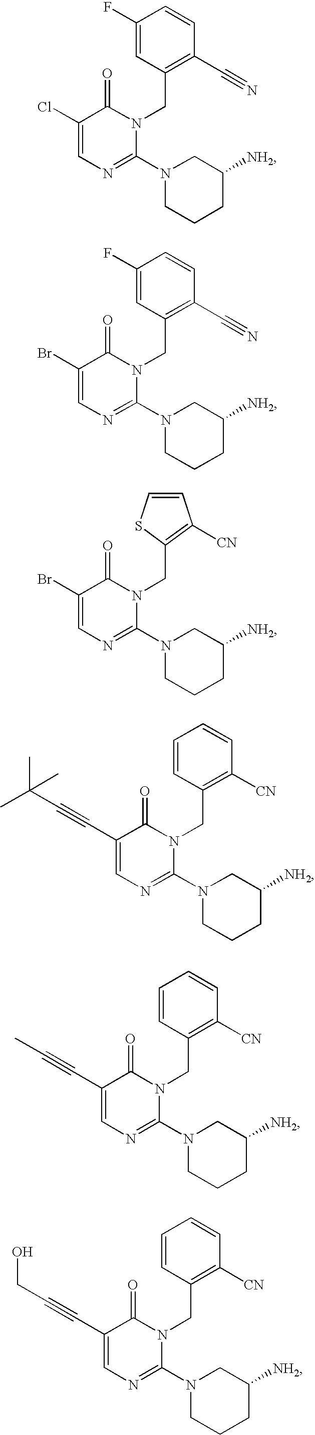

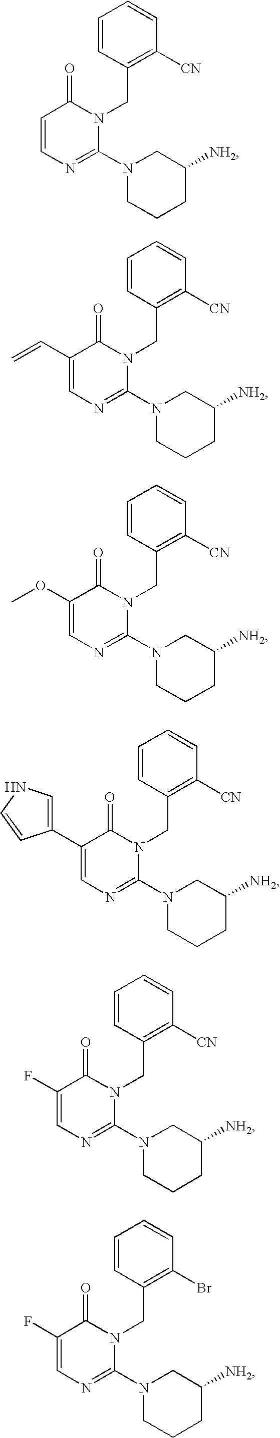

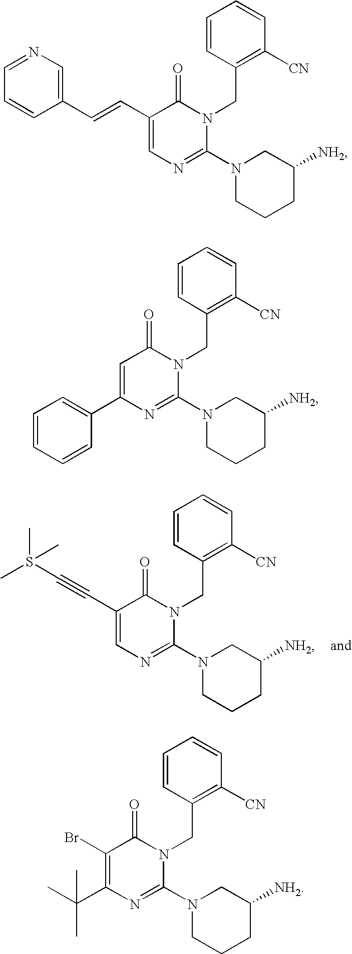

In still further or alternative embodiments, the pyrimidine derivative comprises a compound of the formula:

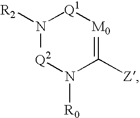

wherein:
MO is -C-LX, N or CR₄;
Z′ is a leaving group;
Q¹ and Q² are each independently selected from the group consisting of CO, CS, SO, SO₂, and C═NR₉;
R₀ is R₁ or -LX, with the proviso that only one of R₀ and M₀ is -LX;
R₁ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₂ is hydrogen or selected from the group consisting of (C₁₋₁₀)alkyl, (C₃₋₁₂)cycloalkyl, (C₃₋₁₂)cycloalkyl(C₁₋₅)alkyl, hetero(C₃₋₁₂)cycloalkyl(C₁₋₅)alkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, hetero(C₄₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl group, imino group, sulfonyl group and sulfinyl group, each substituted or unsubstituted;
R₄ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₉ is hydrogen or is selected from the group consisting of alkyl, cycloalkyl, heterocycloalkyl, arylalkyl, heteroarylalkyl, bicycloaryl, and heterobicycloaryl, each substituted or unsubstituted;
L is a linker providing 1, 2 or 3 atom separation between X and the ring to which L is attached, wherein the atoms of the linker providing the separation are selected from the group consisting of carbon, oxygen, nitrogen, and sulfur; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

In a further or alternative embodiment of these compounds, the Z′ is a halogen, also referred to as halo. In a still further or alternative embodiment, the reaction of the (R)-3-aminopiperidine dihydrochloride, produced by the above process, with the pyrimidine derivative provides a product comprising a compound having the structure according to the following formula,

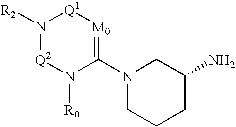

wherein:
M₀ is -C-LX, N or CR₄;
Q¹ and Q² are each independently selected from the group consisting of CO, CS, SO, SO₂, and C═NR₉;
R₀ is R₁ or -LX, with the proviso that only one of R₀ and M₀ is -LX;
R₁ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₂ is hydrogen or selected from the group consisting of (C₁₋₁₀)alkyl, (C₃₋₁₂)cycloalkyl, (C₃₋₁₂)cycloalkyl(C₁₋₅)alkyl, hetero(C₃₋₁₂)cycloalkyl(C₁₋₅)alkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, hetero(C₄₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl group, imino group, sulfonyl group and sulfinyl group, each substituted or unsubstituted;
R₄ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₉ is hydrogen or is selected from the group consisting of alkyl, cycloalkyl, heterocycloalkyl, arylalkyl, heteroarylalkyl, bicycloaryl, and heterobicycloaryl, each substituted or unsubstituted;
L is a linker providing 1, 2 or 3 atom separation between X and the ring to which L is attached, wherein the atoms of the linker providing the separation are selected from the group consisting of carbon, oxygen, nitrogen, and sulfur; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

In even further or alternative embodiments, the reaction of the (R)-3-aminopiperidine dihydrochloride, produced by the above process, with the pyrimidine derivative provides a product comprising a compound selected from the group consisting of

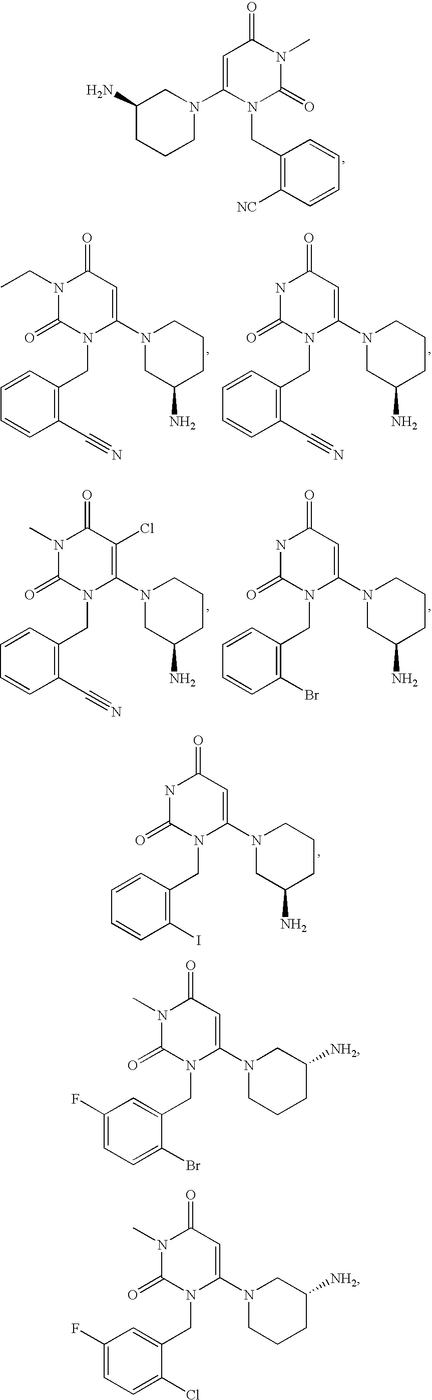

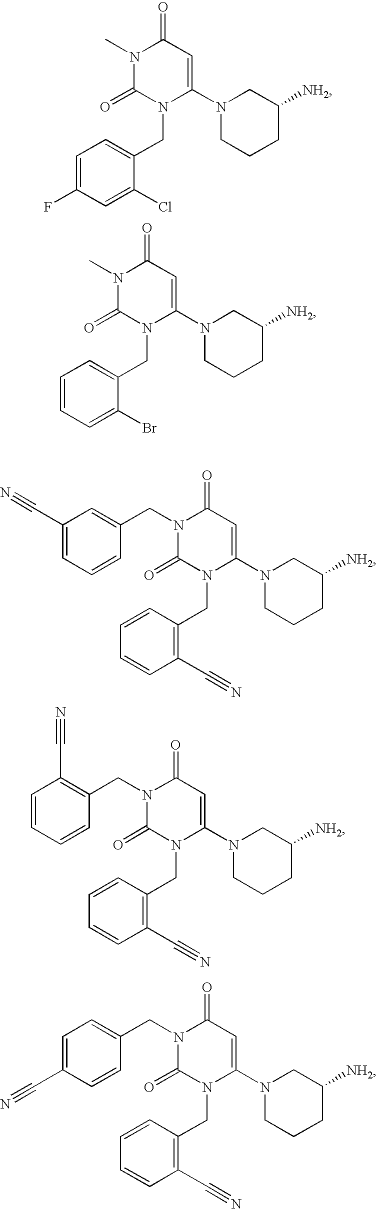

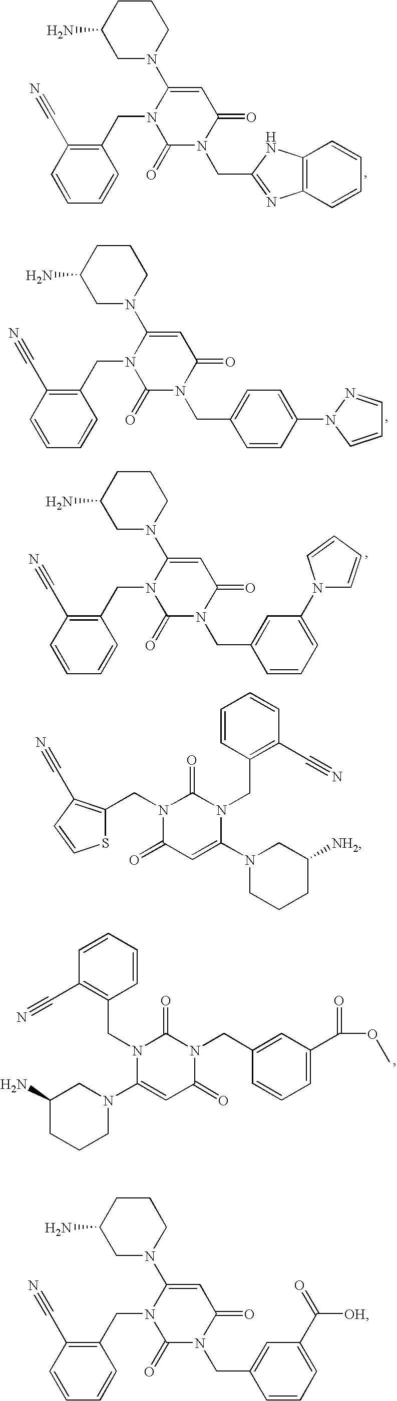

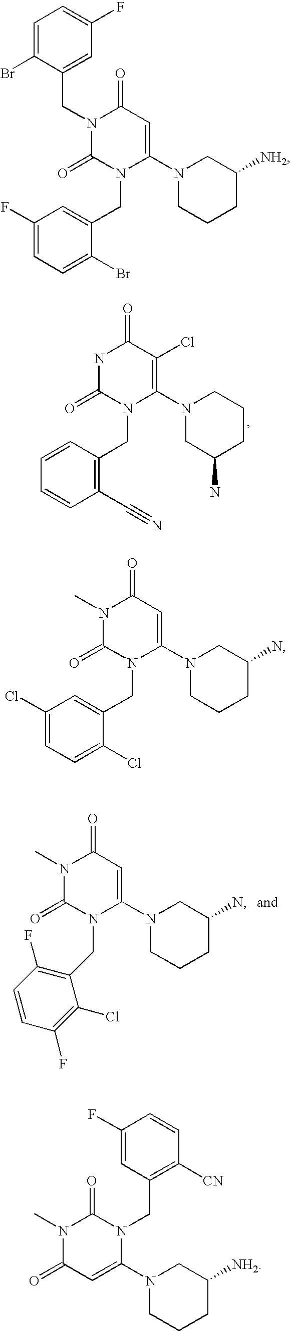

These and other aspects of the methods and compositions described herein will be evident upon reference to the following detailed description.

### Certain Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with (C₃₋₈) rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkenyl” means a straight or branched, carbon chain that contains at least one carbon-carbon double bond (—CR═CR′— or —CR═CR′R″, wherein R, R′ and R″ are each independently hydrogen or further substituents). Examples of alkenyl include vinyl, allyl, isopropenyl, pentenyl, hexenyl, heptenyl, 1-propenyl, 2-butenyl, 2-methyl-2-butenyl, and the like. In particular embodiments, “alkenyl,” either alone or represented along with another radical, can be a (C₂₋₂₀)alkenyl, a (C₂₋₁₅)alkenyl, a (C₂₋₁₀)alkenyl, a (C₂₋₅)alkenyl or a (C₂₋₃)alkenyl. Alternatively, “alkenyl,” either alone or represented along with another radical, can be a (C₂)alkenyl, a (C₃)alkenyl or a (C₄)alkenyl.

“Alkenylene” means a straight or branched, divalent carbon chain having one or more carbon-carbon double bonds (—CR═CR′—, wherein R and R′ are each independently hydrogen or further substituents). Examples of alkenylene include ethene-1,2-diyl, propene-1,3-diyl, methylene-1,1-diyl, and the like. In particular embodiments, “alkenylene,” either alone or represented along with another radical, can be a (C₂₋₂₀) alkenylene, a (C₂₋₁₅) alkenylene, a (C₂₋₁₀) alkenylene, a (C₂₋₅) alkenylene or a (C₂₋₃) alkenylene. Alternatively, “alkenylene,” either alone or represented along with another radical, can be a (C₂) alkenylene, a (C₃) alkenylene or a (C₄) alkenylene.

“Alkoxy” means an oxygen moiety having a further alkyl substituent. The alkoxy groups of the present invention can be optionally substituted.

“Alkyl” represented by itself means a straight or branched, saturated or unsaturated, aliphatic radical having a chain of carbon atoms, optionally with one or more of the carbon atoms being replaced with oxygen (See “oxaalkyl”), a carbonyl group (See “oxoalkyl), sulfur (See “thioalkyl”), and/or nitrogen (See “azaalkyl”). (CX)alkyl and (CX—Y)alkyl are typically used where X and Y indicate the number of carbon atoms in the chain. For example, (C₁₋₆)alkyl includes alkyls that have a chain of between 1 and 6 carbons (e.g., methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl and the like) means a straight or branched, saturated or unsaturated aliphatic divalent radical having the number of atoms indicated or when no atoms are indicated means a bond (e.g., (C6-10)aryl(C1-3)alkyl includes, benzyl, phenethyl, 1-phenylethyl, 3-phenylpropyl, 2-thienylmethyl, 2-pyridinylmethyl and the like). In particular embodiments, “alkyl,” either alone or represented along with another radical, can be a (C1-20)alkyl, a (C1-15)alkyl, a (C1-10)alkyl, a (C1-5)alkyl or a (C1-3)alkyl. Alternatively, “alkyl,” either alone or represented along with another radical, can be a (C1)alkyl, a (C2)alkyl or a (C3)alkyl.

“Alkylene”, unless indicated otherwise, means a straight or branched, saturated or unsaturated, aliphatic, divalent radical. (CX)alkylene and (CX—Y)alkylene are typically used where X and Y indicate the number of carbon atoms in the chain. For example, (C1-6)alkylene includes methylene (—CH2—), ethylene (—CH2CH2—), trimethylene (—CH2CH2CH2—), tetramethylene (—CH2CH2CH2CH2—) 2-butenylene (—CH2CH═CHCH2—), 2-methyltetramethylene (—CH2CH(CH3)CH2CH2—), pentamethylene (—CH2CH2CH2CH2CH2—) and the like. In particular embodiments, “alkylene,” either alone or represented along with another radical, can be a (C1-20)alkylene, a (C1-15)alkylene, a (C1-10)alkylene, a (C1-5)alkylene or a (C1-3)alkylene. Alternatively, “alkylene,” either alone or represented along with another radical, can be a (C1)alkylene, a (C2)alkylene or a (C3)alkylene.

“Alkylidene” means a straight or branched, saturated or unsaturated, aliphatic radical connected to the parent molecule by a double bond. (CX)alkylidene and (CX—Y)alkylidene are typically used where X and Y indicate the number of carbon atoms in the chain. For example, (C1-6)alkylidene includes methylene (═CH2), ethylidene (═CHCH3), isopropylidene (═C(CH3)2), propylidene (═CHCH2CH3), allylidene (═CH—CH═CH2), and the like. In particular embodiments, “alkylidene,” either alone or represented along with another radical, can be a (C1-20)alkylidene, a (C1-15)alkylidene, a (C1-10)alkylidene, a (C1-5)alkylidene or a (C1-3)alkylidene. Alternatively, “alkylidene,” either alone or represented along with another radical, can be a (C1)alkylidene, a (C2)alkylidene or a (C3)alkylidene.

“Alkynyl” means a straight or branched, carbon chain that contains at least one carbon-carbon triple bond (—C≡C— or —C≡CR, wherein R is hydrogen or a further substituent). Examples of alkynyl include ethynyl, propargyl, 3-methyl-l-pentynyl, 2-heptynyl and the like. In particular embodiments, “alkynyl,” either alone or represented along with another radical, can be a (C2-20)alkynyl, a (C2-15)alkynyl, a (C2-10)alkynyl, a (C2-5)alkynyl or a (C2-3)alkynyl. Alternatively, “alkynyl,” either alone or represented along with another radical, can be a (C2)alkynyl, a (C3)alkynyl or a (C4)alkynyl.

“Alkynylene” means a straight or branched, divalent carbon chain having one or more carbon-carbon triple bonds (—CR≡CR′—, wherein R and R′ are each independently hydrogen or further substituents). Examples of alkynylene include ethyne-1,2-diyl, propyne-1,3-diyl, and the like. In particular embodiments, “alkynylene,” either alone or represented along with another radical, can be a (C2-20) alkynylene, a (C2-15) alkynylene, a (C2-10) alkynylene, a (C2-5) alkynylene or a (C2-3) alkynylene. Alternatively, “alkynylene,” either alone or represented along with another radical, can be a (C2) alkynylene, a (C3) alkynylene or a (C4) alkynylene.

“Amino” means a nitrogen moiety having two further substituents where, for example, a hydrogen or carbon atom is attached to the nitrogen. For example, representative amino groups include —NH2, —NHCH3, —N(CH3)2, —NH((C1-10)alkyl), —N((C1-10)alkyl)2, —NH(aryl), —NH(heteroaryl), —N(aryl)2, —N(heteroaryl)2, and the like. Optionally, the two substituents together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aminoalkyl” means an alkyl, as defined above, except where one or more substituted or unsubstituted nitrogen atoms (—N—) are positioned between carbon atoms of the alkyl. For example, an (C2-6) aminoalkyl refers to a chain comprising between 2 and 6 carbons and one or more nitrogen atoms positioned between the carbon atoms.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (See “heteroaryl”).

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. (CX)aryl and (CX—Y)aryl are typically used where X and Y indicate the number of carbon atoms in the ring. In particular embodiments, “aryl,” either alone or represented along with another radical, can be a (C3-14)aryl, a (C3-10)aryl, a (C3-7)aryl, a (C8-10)aryl or a (C5-7)aryl. Alternatively, “aryl,” either alone or represented along with another radical, can be a (C5)aryl, a (C6)aryl, a (C7)aryl, a (C8)aryl, a (C9)aryl or a (C10)aryl.

“Azaalkyl” means an alkyl, as defined above, except where one or more of the carbon atoms forming the alkyl chain are replaced with substituted or unsubstituted nitrogen atoms (—NR— or —NRR′, wherein R and R′ are each independently hydrogen or further substituents). For example, a (C1-10)azaalkyl refers to a chain comprising between 1 and 10 carbons and one or more nitrogen atoms.

“Bicycloalkyl” means a saturated or partially unsaturated fused, spiro or bridged bicyclic ring assembly. In particular embodiments, “bicycloalkyl,” either alone or represented along with another radical, can be a (C4-15)bicycloalkyl, a (C4-10)bicycloalkyl, a (C6-10)bicycloalkyl or a (C8-10)bicycloalkyl. Alternative “bicycloalkyl,” either alone or represented along with another radical, can be a (C8)bicycloalkyl, a (C9)bicycloalkyl or a (C10)bicycloalkyl.

“Bicycloaryl” means a fused, spiro or bridged bicyclic ring assembly wherein at least one of the rings comprising the assembly is aromatic. (CX)bicycloaryl and (CX—Y)bicycloaryl are typically used where X and Y indicate the number of carbon atoms in the bicyclic ring assembly and directly attached to the ring. In particular embodiments, “bicycloaryl,” either alone or represented along with another radical, can be a (a (C4-15)bicycloaryl, a (C4-10)bicycloaryl, a (C6-10)bicycloaryl or a (C8-10)bicycloaryl. Alternatively, “bicycloalkyl,” either alone or represented along with another radical, can be a (C8)bicycloaryl, a (C9)bicycloaryl or a (C10)bicycloaryl.

“Bridging ring” and “bridged ring” as used herein refer to a ring that is bonded to another ring to form a compound having a bicyclic or polycyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRR′, wherein R and R′ are each independently hydrogen or further substituents.

“Carbocycle” means a ring consisting of carbon atoms.

“Carbonyl” means the radical —C(═O)— and/or —C(═O)R, wherein R is hydrogen or a further substituent. It is noted that the carbonyl radical may be further substituted with a variety of substituents to form different carbonyl groups including acids, acid halides, aldehydes, amides, esters, and ketones.

“Carboxy” means the radical —C(═O)—O— and/or —C(═O)—OR, wherein R is hydrogen or a further substituent. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a non-aromatic, saturated or partially unsaturated, monocyclic, bicyclic or polycyclic ring assembly. (CX)cycloalkyl and (CX—Y)cycloalkyl are typically used where X and Y indicate the number of carbon atoms in the ring assembly. For example, (C3-10)cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like. In particular embodiments, “cycloalkyl,” either alone or represented along with another radical, can be a (C3-14)cycloalkyl, a (C3-10)cycloalkyl, a (C3-7)cycloalkyl, a (C8-10)cycloalkyl or a (C5-7)cycloalkyl. Alternatively, “cycloalkyl,” either alone or represented along with another radical, can be a (C5)cycloalkyl, a (C6)cycloalkyl, a (C7)cycloalkyl, a (C8)cycloalkyl., a (C9)cycloalkyl or a (C10)cycloalkyl.

“Cycloalkylene” means a divalent, saturated or partially unsaturated, monocyclic, bicyclic or polycyclic ring assembly. (CX)cycloalkylene and (CX—Y)cycloalkylene are typically used where X and Y indicate the number of carbon atoms in the ring assembly. In particular embodiments, “cycloalkylene,” either alone or represented along with another radical, can be a (C3-14)cycloalkylene, a (C3-10)cycloalkylene, a (C3-7)cycloalkylene, a (C8-10)cycloalkylene or a (C5-7)cycloalkylene. Alternatively, “cycloalkylene,” either alone or represented along with another radical, can be a (C5)cycloalkylene, a (C6)cycloalkylene, a (C7)cycloalkylene, a (C8)cycloalkylene, a (C9)cycloalkylene or a (C10)cycloalkylene.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a ring that is bonded to another ring to form a compound having a bicyclic structure where the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems may be saturated, partially saturated, carbocyclics, heterocyclics, aromatics, heteroaromatics, and the like.

“Halo” and “halogen” mean fluoro, chloro, bromo or iodo.

“Heteroalkyl” means alkyl, as defined in this Application, provided that one or more of the atoms within the alkyl chain is a heteroatom. In particular embodiments, “heteroalkyl,” either alone or represented along with another radical, can be a hetero(C1-20)alkyl, a hetero(C1-15)alkyl, a hetero(C1-10)alkyl, a hetero(C1-5)alkyl, a hetero(C1-3)alkyl or a hetero(C1-2)alkyl. Alternatively, “heteroalkyl,” either alone or represented along with another radical, can be a hetero(C1l)alkyl, a hetero(C2)alkyl or a hetero(C3)alkyl.

“Heteroaryl” means a monocyclic, bicyclic or polycyclic aromatic group wherein at least one ring atom is a heteroatom and the remaining ring atoms are carbon. Monocyclic heteroaryl groups include, but are not limited to, cyclic aromatic groups having five or six ring atoms, wherein at least one ring atom is a heteroatom and the remaining ring atoms are carbon. The nitrogen atoms can be optionally quaternerized and the sulfur atoms can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, a cycloalkenyl ring, and another monocyclic heteroaryl or heterocycloalkyl ring. These bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo [1,5-a]pyridine, imidazo [1,2-a]pyrimidine, imidazo [1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The bicyclic or tricyclic heteroaryl rings can be attached to the parent molecule through either the heteroaryl group itself or the aryl, cycloalkyl, cycloalkenyl or heterocycloalkyl group to which it is fused. The heteroaryl groups of this invention can be substituted or unsubstituted. In particular embodiments, “heteroaryl,” either alone or represented along with another radical, can be a hetero(C1-13)aryl, a hetero(C2-13)aryl, a hetero(C2-6)aryl, a hetero(C3-9)aryl or a hetero(C5-9)aryl. Alternatively, “heteroaryl,” either alone or represented along with another radical, can be a hetero(C3)aryl, a hetero(C4)aryl, a hetero(C5)aryl, a hetero(C6)aryl., a hetero(C7)aryl, a hetero(C8)aryl or a hetero(C9)aryl.

“Heteroatom” refers to an atom that is not a carbon atom. Particular examples of heteroatoms include, but are not limited to, nitrogen, oxygen, and sulfur.

“Heteroatom moiety” includes a moiety where the atom by which the moiety is attached is not a carbon. Examples of heteroatom moieties include —NR—, —N═(O—)═, —O—, —S— or —S(O)2—, wherein R is hydrogen or a further substituent.

“Heterobicycloalkyl” means bicycloalkyl, as defined in this Application, provided that one or more of the atoms within the ring is a heteroatom. For example hetero(C9-12)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex- and the like. In particular embodiments, “heterobicycloalkyl,” either alone or represented along with another radical, can be a hetero(C1-14)bicycloalkyl, a hetero(C4-14)bicycloalkyl, a hetero(C4-9)bicycloalkyl or a hetero(C5-9)bicycloalkyl. Alternatively, “heterobicycloalkyl,” either alone or represented along with another radical, can be a hetero(C5)bicycloalkyl, hetero(C6)bicycloalkyl, hetero(C7)bicycloalkyl, hetero(C8)bicycloalkyl or a hetero(C9)bicycloalkyl.

“Heterobicycloaryl” means bicycloaryl, as defined in this Application, provided that one or more of the atoms within the ring is a heteroatom. For example, hetero(C4-12)bicycloaryl as used in this Application includes, but is not limited to, 2-amino-4-oxo-3,4-dihydropteridin-6-yl, tetrahydroisoquinolinyl, and the like. In particular embodiments, “heterobicycloaryl,” either alone or represented along with another radical, can be a hetero(C1-14)bicycloaryl, a hetero(C4-14)bicycloaryl, a hetero(C4-9)bicycloarylor a hetero(C5-9)bicycloaryl. Alternatively, “heterobicycloaryl,” either alone or represented along with another radical, can be a hetero(C5)bicycloaryl, hetero(C6)bicycloaryl, hetero(C7)bicycloaryl, hetero(C8)bicycloaryl or a hetero(C9)bicycloaryl.

“Heterocycloalkyl” means cycloalkyl, as defined in this Application, provided that one or more of the atoms forming the ring is a heteroatom selected, independently from N, O, or S. Non-exclusive examples of heterocycloalkyl include piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like. In particular embodiments, “heterocycloalkyl,” either alone or represented along with another radical, can be a hetero(C1-1 3)cycloalkyl, a hetero(C1-9)cycloalkyl, a hetero(C1-6)cycloalkyl, a hetero(C5-9)cycloalkyl or a hetero(C2-6)cycloalkyl. Alternatively, “heterocycloalkyl,” either alone or represented along with another radical, can be a hetero(C2)cycloalkyl, a hetero(C3)cycloalkyl, a hetero(C4)cycloalkyl, a hetero(C5)cycloalkyl, a hetero(C6)cycloalkyl, hetero(C7)cycloalkyl, hetero(C8)cycloalkyl or a hetero(C9)cycloalkyl.

“Heterocycloalkylene” means cycloalkylene, as defined in this Application, provided that one or more of the ring member carbon atoms is replaced by a heteroatom. In particular embodiments, “heterocycloalkylene,” either alone or represented along with another radical, can be a hetero(C1-1 3)cycloalkylene, a hetero(C1-9)cycloalkylene, a hetero(C1-6)cycloalkylene, a hetero(C5-9)cycloalkylene or a hetero(C2-6)cycloalkylene. Alternatively, “heterocycloalkylene,” either alone or represented along with another radical, can be a hetero(C2)cycloalkylene, a hetero(C3)cycloalkylene, a hetero(C4)cycloalkylene, a hetero(C5)cycloalkylene, a hetero(C6)cycloalkylene, hetero(C7)cycloalkylene, hetero(C8)cycloalkylene or a hetero(C9)cycloalkylene.

“Hydroxy” means the radical —OH.

“Imino” means the radical —CR(═NR′) and/or —C(═NR′)—, wherein R and R′ are each independently hydrogen or a further substituent.

“Isomers” means compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.” A compound that has more than one chiral center has 2n+1 enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Leaving group” means the group with the meaning conventionally associated with it in synthetic organic chemistry, i.e., an atom or group displaceable under reaction (e.g., alkylating) conditions. Examples of leaving groups include, but are not limited to, halo (e.g., F, Cl, Br and I), alkyl (e.g., methyl and ethyl) and sulfonyloxy (e.g., mesyloxy, ethanesulfonyloxy, benzenesulfonyloxy and tosyloxy), thiomethyl, thienyloxy, dihalophosphinoyloxy, tetrahalophosphoxy, benzyloxy, isopropyloxy, acyloxy, and the like.

“Moiety providing X atom separation” and “linker providing X atom separation” between two other moieties mean that the chain of atoms directly linking the two other moieties is X atoms in length. When X is given as a range (e.g., X1-X2), then the chain of atoms is at least X1 and not more than X2 atoms in length. It is understood that the chain of atoms can be formed from a combination of atoms including, for example, carbon, nitrogen, sulfur and oxygen atoms. Further, each atom can optionally be bound to one or more substituents, as valencies allow. In addition, the chain of atoms can form part of a ring. Accordingly, in one embodiment, a moiety providing X atom separation between two other moieties (R and R′) can be represented by R-(L)X—R′ where each L is independently selected from the group consisting of CR″R′″, NR″″, O, S, CO, CS, C═NR′″″, SO, SO₂, and the like, where any two or more of R″, R′″, R″″ and R′″″ can be taken together to form a substituted or unsubstituted ring.

“Nitro” means the radical —NO2.

“Oxaalkyl” means an alkyl, as defined above, except where one or more of the carbon atoms forming the alkyl chain are replaced with oxygen atoms (—O— or —OR, wherein R is hydrogen or a further substituent). For example, an oxa(C1-10)alkyl refers to a chain comprising between 1 and 10 carbons and one or more oxygen atoms.

“Oxoalkyl” means an alkyl, as defined above, except where one or more of the carbon atoms forming the alkyl chain are replaced with carbonyl groups (—C(═O)— or —C(═O)—R, wherein R is hydrogen or a further substituent). The carbonyl group may be an aldehyde, ketone, ester, amide, acid or acid halide. For example, an oxo(C1-10)alkyl refers to a chain comprising between 1 and 10 carbon atoms and one or more carbonyl groups.

“Oxy” means the radical —O— or —OR, wherein R is hydrogen or a further substituent. Accordingly, it is noted that the oxy radical may be further substituted with a variety of substituents to form different oxy groups including hydroxy, alkoxy, aryloxy, heteroaryloxy or carbonyloxy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Polycyclic ring” includes bicyclic and multi-cyclic rings. The individual rings comprising the polycyclic ring can be fused, spiro or bridging rings.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

“Protected derivatives” means derivatives of inhibitors in which a reactive site or sites are blocked with protecting groups. Protected derivatives are useful in the preparation of inhibitors or in themselves may be active as inhibitors. A comprehensive list of suitable protecting groups can be found in T.W. Greene, Protecting Groups in Organic Synthesis, 3rd edition, John Wiley & Sons, Inc. 1999.

“Ring” and “ring assembly” means a carbocyclic or a heterocyclic system and includes aromatic and non-aromatic systems. The system can be monocyclic, bicyclic or polycyclic. In addition, for bicyclic and polycyclic systems, the individual rings comprising the polycyclic ring can be fused, spiro or bridging rings.

“Subject” and “patient” includes humans, non-human mammals (e.g., dogs, cats, rabbits, cattle, horses, sheep, goats, swine, deer, and the like) and non-mammals (e.g., birds, and the like).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, t-butoxycarbonyl [(CH3)3C—OCO—], benzyloxycarbonyl, p-methoxybenzyloxycarbonyl, vinyloxycarbonyl, β-(p-toluenesulfonyl)ethoxycarbonyl, and the like. Examples of suitable amino acid residues include amino acid residues per se and amino acid residues that are protected with a protecting group. Suitable amino acid residues include, but are not limited to, residues of Gly (glycine), Ala (alanine; CH3CH(NH2)CO—), Arg (arginine), Asn (asparagine), Asp (aspartic acid), Cys (cysteine), Glu (glutamic acid), His (histidine), Ile (isoleucine), Leu (leucine; (CH3)2CHCH2CH(NH2)CO—), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH3)3C—OCO—], and the like. Suitable peptide residues include peptide residues comprising two to five, and optionally two to three, of the aforesaid amino acid residues. Examples of such peptide residues include, but are not limited to, residues of such peptides as Ala-Ala [CH3CH(NH2)CO—NHCH(CH3)CO—], Gly-Phe, Nva-Nva, Ala-Phe, Gly-Gly, Gly-Gly-Gly, Ala-Met, Met-Met, Leu-Met and Ala-Leu. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH3)3C—OCO—], and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups (such as β,β,β-trichloroethoxycarbonyl and β-iodoethoxycarbonyl).

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃ In general, a non-hydrogen substituent may be any substituent that may be bound to an atom of the given moiety that is specified to be substituted. Examples of substituents include, but are not limited to, aldehyde, alicyclic, aliphatic, (C₁₋₁₀)alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted. In one particular embodiment, examples of substituents include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(Cl lo)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(Cl lo)alkyl, aryl(Cl lo)alkyl, hetero(Cl lo)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl.

“Sulfinyl” means the radical —SO— and/or —SO—R, wherein R is hydrogen or a further substituent. It is noted that the sulfinyl radical may be further substituted with a variety of substituents to form different sulfinyl groups including sulfinic acids, sulfinamides, sulfinyl esters, and sulfoxides.

“Sulfonyl” means the radical —SO₂— and/or —SO₂—R, wherein R is hydrogen or a further substituent. It is noted that the sulfonyl radical may be further substituted with a variety of substituents to form different sulfonyl groups including sulfonic acids, sulfonamides, sulfonate esters, and sulfones.

“Therapeutically effective amount” means that amount which, when administered to an animal for treating a disease, is sufficient to effect such treatment for the disease.

“Thio” denotes replacement of an oxygen by a sulfur and includes, but is not limited to, —SR, —S— and ═S containing groups.

“Thioalkyl” means an alkyl, as defined above, except where one or more of the carbon atoms forming the alkyl chain are replaced with sulfur atoms (—S— or —S—R, wherein R is hydrogen or a further substituent). For example, a thio(C₁₋₁₀)alkyl refers to a chain comprising between 1 and 10 carbons and one or more sulfur atoms.

“Thiocarbonyl” means the radical —C(═S)— and/or —C(═S)—R, wherein R is hydrogen or a further substituent. It is noted that the thiocarbonyl radical may be further substituted with a variety of substituents to form different thiocarbonyl groups including thioacids, thioamides, thioesters, and thioketones.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a (C₁)alkyl comprises methyl (i.e., —CH₃) as well as —CRR′R″ where R, R′, and R″ may each independently be hydrogen or a further substituent where the atom attached to the carbon is a heteroatom or cyano. Hence, CF₃, CH₂OH and CH₂CN, for example, are all (C₁)alkyls. Similarly, terms such as alkylamino and the like comprise dialkylamino and the like.

As used herein the symbols and conventions used in these processes, schemes and examples described herein are consistent with those used in the contemporary scientific literature, for example, the Journal of the American Chemical Society or the Journal of Biological Chemistry. Standard single-letter or thee-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification: g (grams); L (liters); mL (milliliters); min (minutes); h (hours); RT (ambient temperature); MeOH (methanol); THF (tetrahydrofuran); Ac (acetyl); TLC (thin layer chromatography); MTBE (methyl tert-butyl ether); HPLC (high pressure liquid chomatography), and unless otherwise indicated, all temperatures are expressed in ° C. (degrees Centigrade).

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description. All references cited herein, including patents, patent applications, and publications, are hereby incorporated by reference in their entirety.

### DETAILED DESCRIPTION OF THE INVENTION

Dipeptidyl Peptidase IV (IUBMB Enzyme Nomenclature EC.3.4.14.5) is a type II membrane protein that has been referred to in the literature by a wide a variety of names including DPP4, DP4, DAP-IV, FAPβ, adenosine deaminase complexing protein 2, adenosine deaminase binding protein (ADAbp), dipeptidyl aminopeptidase IV; Xaa-Pro-dipeptidyl-aminopeptidase; Gly-Pro naphthylamidase; postproline dipeptidyl aminopeptidase IV; lymphocyte antigen CD26; glycoprotein GP110; dipeptidyl peptidase IV; glycylproline aminopeptidase; glycylproline aminopeptidase; X-prolyl dipeptidyl aminopeptidase; pep X; leukocyte antigen CD26; glycylprolyl dipeptidylaminopeptidase; dipeptidyl-peptide hydrolase; glycylprolyl aminopeptidase; dipeptidyl-aminopeptidase IV; DPP IV/CD26; amino acyl-prolyl dipeptidyl aminopeptidase; T cell triggering molecule Tp103; and X-PDAP. Dipeptidyl Peptidase IV is referred to herein as “DPP-IV.”

DPP-IV is a non-classical serine aminodipeptidase that removes Xaa-Pro dipeptides from the amino terminus (N-terminus) of polypeptides and proteins. DPP-IV dependent slow release of dipeptides of the type X-Gly or X-Ser has also been reported for some naturally occurring peptides.

DPP-IV is responsible for the metabolic cleavage of certain endogenous peptides (GLP-1 (7-36), glucagon) in vivo and has demonstrated proteolytic activity against a variety of other peptides (GHRH, NPY, GLP-2, VIP) in vitro. DPP-IV is constitutively expressed on epithelial and endothelial cells of a variety of different tissues (intestine, liver, lung, kidney and placenta), and is also found in body fluids. DPP-IV is also expressed on circulating T-lymphocytes and has been shown to be synonymous with the cell-surface antigen, CD-26.

DPP-IV is believed to contribute to the pathology and/or symptomology of several different diseases such that reduction of the activity of DPP-IV in a subject through inhibition may be used to therapeutically address these disease states. Examples of various conditions or diseases which may be treated or prevented using such DPP-IV inhibitors include, but are not limited to, diabetes, type 2 diabetes mellitus, diabetic dislipidemia, conditions of impaired glucose tolerance (IGT), conditions of impaired fasting plasma glucose (IFG), metabolic acidosis, ketosis, appetite regulation, obesity, various cancers (including breast cancer, lung cancer and prostate cancer), organ transplant rejection; autoimmune diseases such as inflammatory bowel disease, multiple sclerosis and rheumatoid arthritis; and AIDS, dermatological diseases such as psoriasis, rheumatoid arthritis (RA) and lichen planus, infertility and amenorrhea.

DPP-IV inhibitors may also be used to modulate cleavage of various cytokines (stimulating hematopoietic cells), growth factors and neuropeptides. For example, such conditions occur frequently in patients who are immunosuppressed, for example, as a consequence of chemotherapy and/or radiation therapy for cancer. DPP-IV inhibitors may also prevent or reduce cleavage of N-terminal Tyr-Ala from growth hormone-releasing factor. Accordingly, these inhibitors may be used in the treatment of short stature due to growth hormone deficiency (Dwarfism) and for promoting GH-dependent tissue growth or re-growth. Additionally, DPP-IV inhibitors may also address disease states associated with cleavage of neuropeptides and thus may be useful for the regulation or normalization of neurological disorders.

For oncology indications, DPP-IV inhibitors may be used in conjunction with other agents to inhibit undesirable and uncontrolled cell proliferation. Examples of other anti-cell proliferation agents that may be used in conjunction with such DPP-IV inhibitors include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulfate (clupeine), sulfated chitin derivatives (prepared from queen crab shells), sulfated polysaccharide peptidoglycan complex (sp-pg), staurosporine, modulators of matrix metabolism, including for example, proline analogs ((1-azetidine-2-carboxylic acid (LACA)), cishydroxyproline, d,1-3,4-dehydroproline, thiaproline, beta.-aminopropionitrile fumarate, 4-propyl-5-(4-pyridinyl)-2(3H)-oxazolone, methotrexate, mitoxantrone, heparin, interferons, 2 macroglobulin-serum, chimp-3, chymostatin, beta.-cyclodextrin tetradecasulfate, eponemycin; fumagillin, gold sodium thiomalate, d-penicillamine (CDPT), beta.-1-anticollagenase-serum, alpha.2-antiplasmin, bisantrene, lobenzarit disodium, n-2-carboxyphenyl-4-chloroanthronilic acid disodium or “CCA”, thalidomide; angostatic steroid, carboxyaminoimidazole; metalloproteinase inhibitors such as BB94. Other anti-angiogenesis agents that may be used include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-1364.

One class of DPP-IV inhibitors are compounds having the structure of Formula (I),

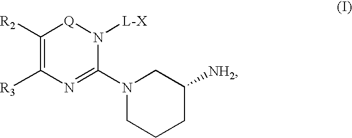

wherein:
Q is selected from the group consisting of CO, SO, SO₂, and C═NR₄;
R₂ and R₃ are each independently selected from the group consisting of hydrogen, halo, perhalo(C₁₋₁₀)alkyl, amino, cyano, nitro, thio, (C₁₋₁₀)alkyl, alkene, alkyne, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₂₋₃)alkyl, imino (C₁₋₃)alkyl, aryl, heteroaryl, hydroxy, alkoxy, aryloxy, heteroaryloxy, alkenyl, alkynyl, carbonyl group, imine group, sulfonyl group and sulfinyl group, each substituted or unsubstituted;

R₄ is selected from the group consisting of hydrogen, (C₁₋₁₀)alkyl, cycloalkyl, heterocycloalkyl, aryl, heteroaryl, arylalkyl, heteroarylalkyl, bicycloaryl, heterobicycloaryl, each substituted or unsubstituted;
L is a linker providing 0-6 atom separation between X and the ring to which L is attached; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

Also described herein are the pro-drugs, pharmaceutically-acceptable salts and solvates of the compound of Formula (I).

The compounds of Formula (I) include a (R)-3-aminopiperidinyl moiety that can be prepared using the methods described herein. The compounds of Formula (I) may be synthesized by methods described in U.S. Patent Application Publication Nos. 2005/0070535, 2005/0070530, 2005/0070706, 2005/0065148, 2005/0070531 and 2005/0075330, and PCT Publication No. WO 2005/016911, the disclosures of which are incorporated by reference in their entirety. For example, compounds of Formula (I) may be prepared by reaction of (R)-3-aminopiperidine dihydrochloride with an appropriate pyrimidine derivative. In one embodiment, the appropriate pyrimidine derivative comprises a compound having the structure:

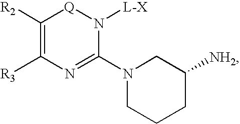

wherein:
Q is selected from the group consisting of CO, SO, SO₂, and C═NR₄;
Z′ is a leaving group;
R₂ and R₃ are each independently selected from the group consisting of hydrogen, halo, perhalo(C₁₋₁₀)alkyl, amino, cyano, nitro, thio, (C₁₋₁₀)alkyl, alkene, alkyne, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl group, imine group, sulfonyl group and sulfinyl group, each substituted or unsubstituted;
R₄ is selected from the group consisting of hydrogen, (C₁₋₁₀)alkyl, cycloalkyl, heterocycloalkyl, aryl, heteroaryl, arylalkyl, heteroarylalkyl, bicycloaryl, heterobicycloaryl, each substituted or unsubstituted;
L is a linker providing 0-6 atom separation between X and the ring to which L is attached; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

In certain embodiments, Z′ is a halo. In further or alternative embodiments, the compound of Formula (I) is produced by the following method:

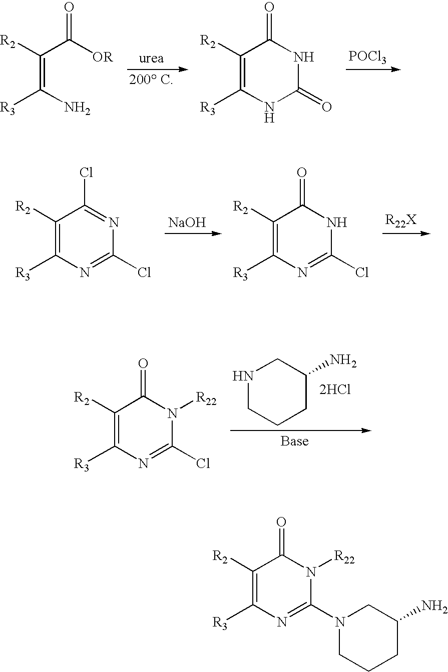

By varying the R₂, R₃, and R₂₂X groups shown above in this example, a wide variety of different DPP-IV inhibitors may be synthesized.

By way of example, (R)-3-aminopiperidine dihydrochloride, as prepared by the methods described herein, may be used in the synthesis of the following DPP-IV inhibitors,

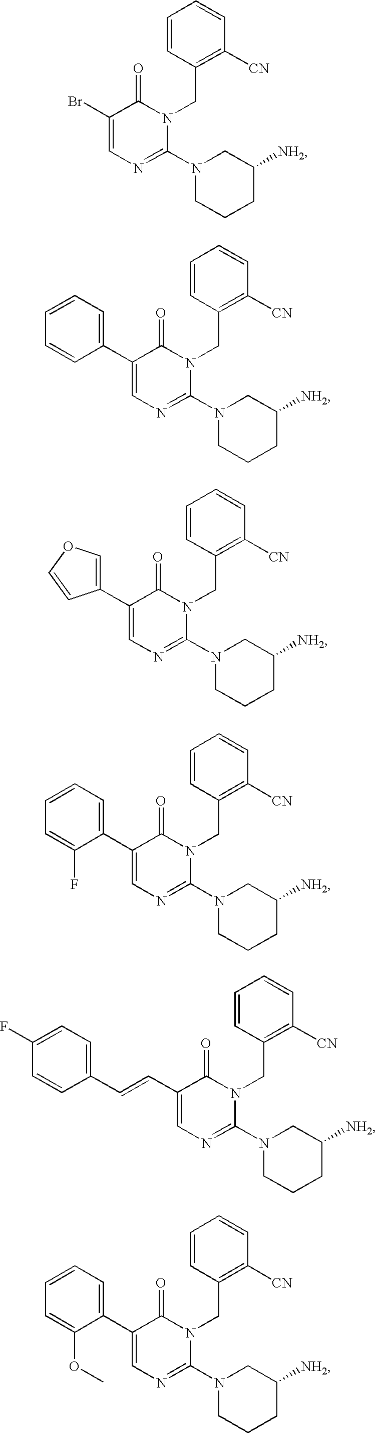

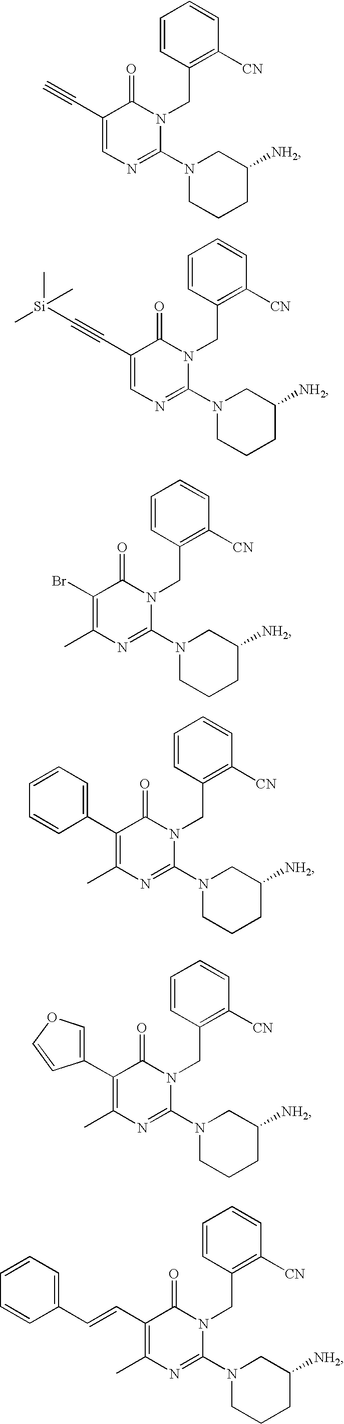

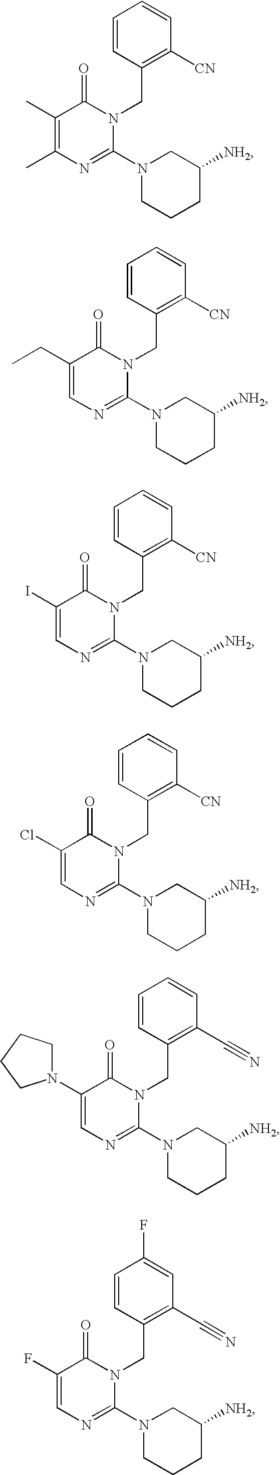

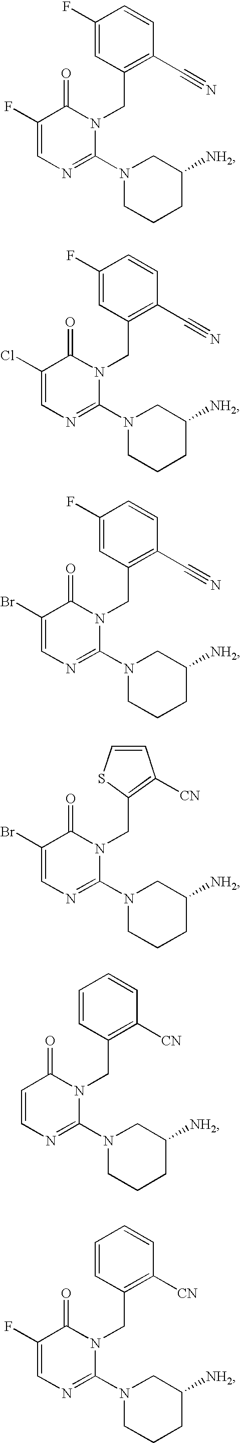

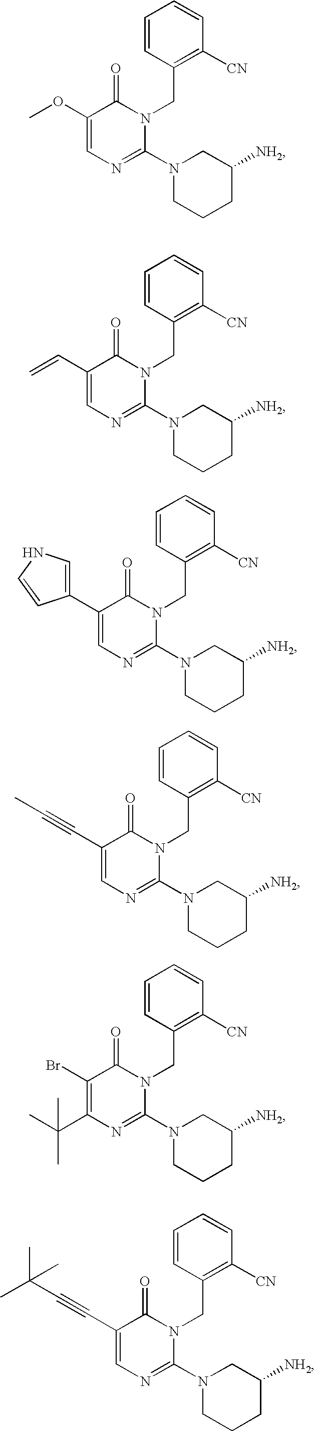

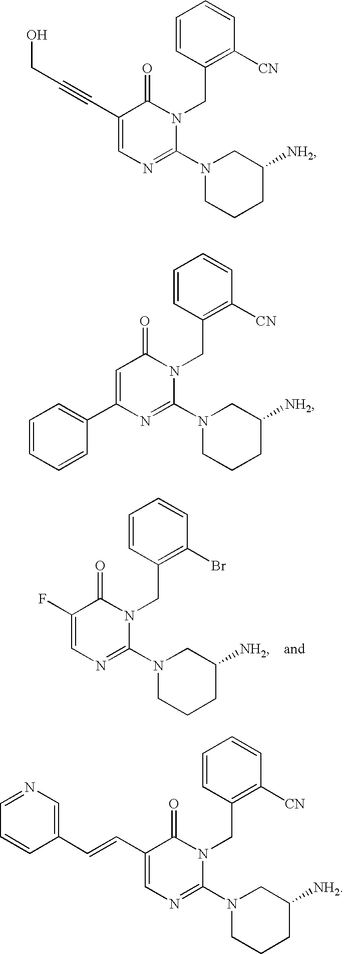

Another class of DPP-IV inhibitors are compounds having the Formula (II),

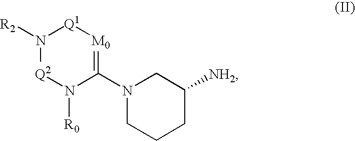

wherein:
M₀ is -C-LX, N or CR₄;
Q¹ and Q² are each independently selected from the group consisting of CO, CS, SO, SO₂, and C═NR₉;
R₀ is R₁ or -LX, with the proviso that only one of R₀ and M₀ is -LX;
R₁ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₂ is hydrogen or selected from the group consisting of (C₁₋₁₀)alkyl, (C₃₋₁₂)cycloalkyl, (C₃₋₁₂)cycloalkyl(C₁₋₅)alkyl, hetero(C₃₋₁₂)cycloalkyl(C₁₋₅)alkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, hetero(C₄₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl group, imino group, sulfonyl group and sulfinyl group, each substituted or unsubstituted;

R₄ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;

R₉ is hydrogen or is selected from the group consisting of alkyl, cycloalkyl, heterocycloalkyl, arylalkyl, heteroarylalkyl, bicycloaryl, and heterobicycloaryl, each substituted or unsubstituted;
L is a linker providing 1, 2 or 3 atom separation between X and the ring to which L is attached, wherein the atoms of the linker providing the separation are selected from the group consisting of carbon, oxygen, nitrogen, and sulfur; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

The compounds of Formula (II) include a (R)-3-aminopiperidinyl moiety that can be prepared using the methods described herein. The compounds of Formula (II) may be synthesized by methods described in U.S. patent application Ser. No. 11/080,992, filed Mar. 15, 2005, and PCT Application Serial No. PCT/US04/42209, filed Dec. 15, 2004, the disclosures of which are incorporated by reference in their entirety. For example, compounds of Formula (II) may be prepared by reaction of (R)-3-aminopiperidine dihydrochloride with an appropriate pyrimidine derivative. In one embodiment, the appropriate pyrimidine derivative comprises a compound having the structure:

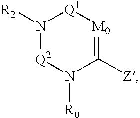

wherein:
M₀ is -C-LX, N or CR₄;
Z′ is a leaving group;
Q¹ and Q² are each independently selected from the group consisting of CO, CS, SO, SO₂, and C═NR₉;
R₀ is R₁ or -LX, with the proviso that only one of R₀ and M₀ is -LX;
R₁ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted; is hydrogen or selected from the group consisting of (C₁₋₁₀)alkyl, (C₃₋₁₂)cycloalkyl, (C₃₋₁₂)cycloalkyl(C₁₋₅)alkyl, hetero(C₃₋₁₂)cycloalkyl(C₁₋₅)alkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, hetero(C₄₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl group, imino group, sulfonyl group and sulfinyl group, each substituted or unsubstituted;
R₄ is hydrogen or is selected from the group consisting of halo, perhalo(C₁₋₁₀o)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;

R₉ is hydrogen or is selected from the group consisting of alkyl, cycloalkyl, heterocycloalkyl, arylalkyl, heteroarylalkyl, bicycloaryl, and heterobicycloaryl, each substituted or unsubstituted;
L is a linker providing 1, 2 or 3 atom separation between X and the ring to which L is attached, wherein the atoms of the linker providing the separation are selected from the group consisting of carbon, oxygen, nitrogen, and sulfur; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

In further embodiments, Z′ is a halo. In further or alternative embodiments, the compound of Formula (II) is produced by the following method:

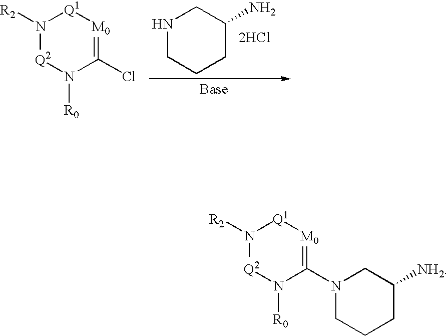

By way of example, the (R)-3-aminopiperidine dihydrochloride, synthesized as described herein, reacts with an appropriate pyrimidine derivative, to produce DPP-IV inhibitors selected from the group:

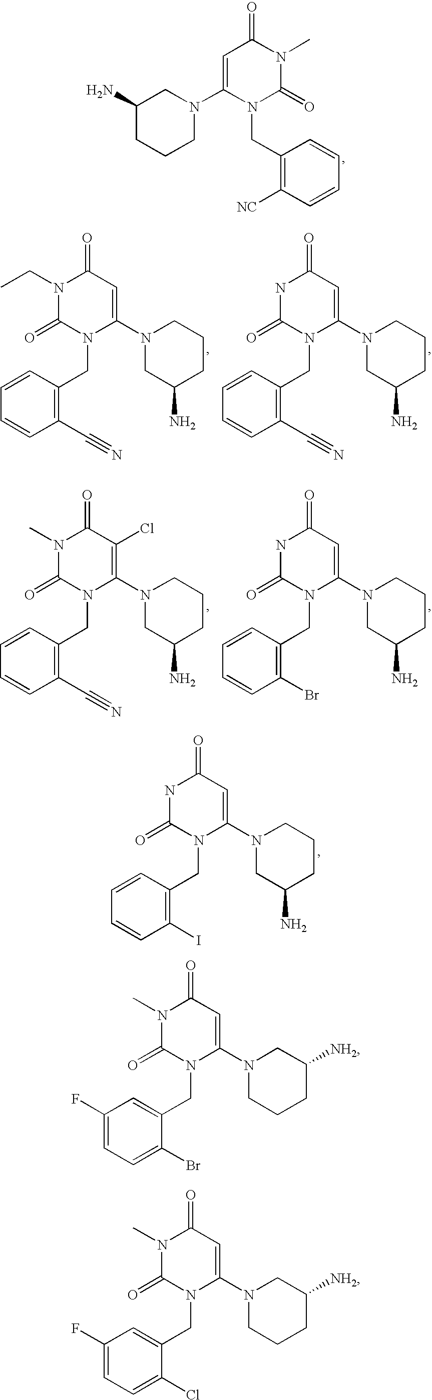

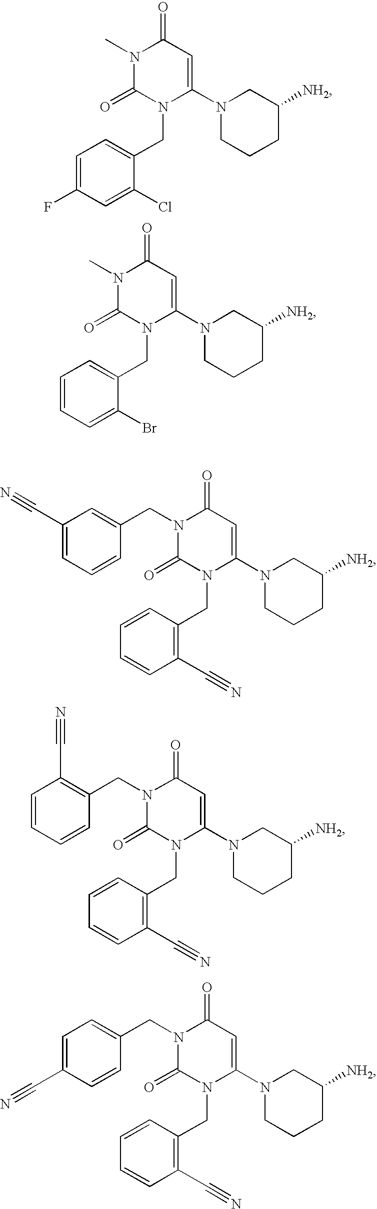

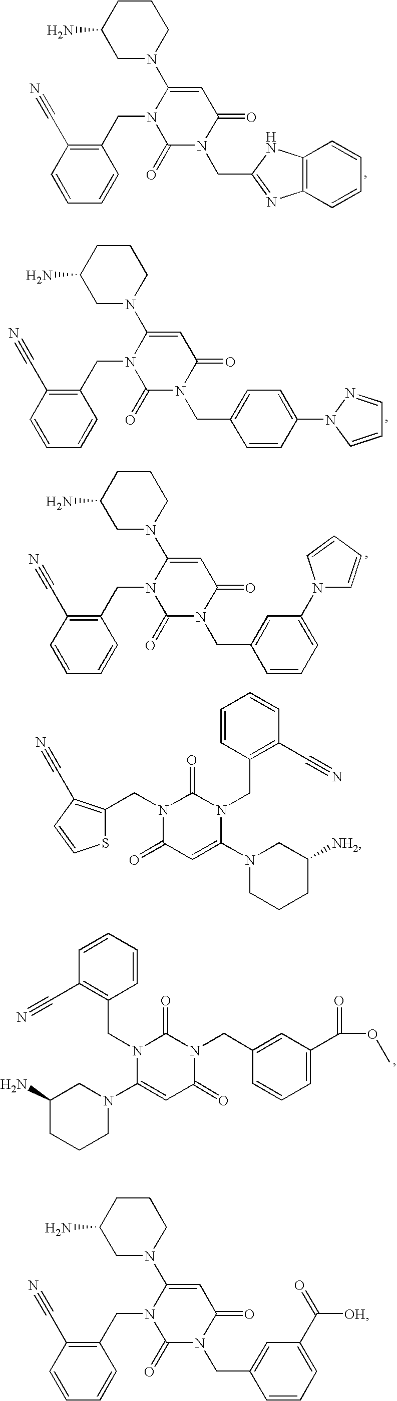

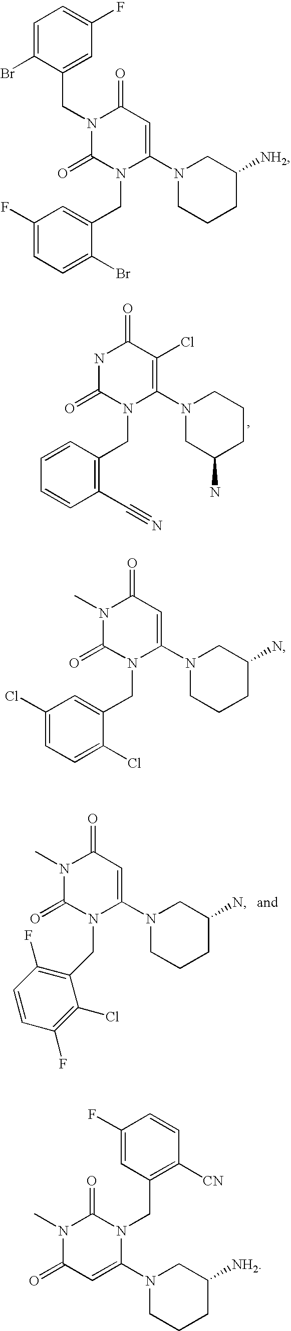

Although the compounds of Formulae (I) and (II) presented above possess an (R)-3-aminopiperidinyl moiety, the methods and techniques described herein may also be used to produce compounds having the opposite enantiomeric configuration, i.e., substitution of an (S)-3-aminopiperidinyl moiety for the (R)-3-aminopiperidinyl moiety. As such, compounds of Formulae (I) and (II) in which an (S)-3-aminopiperidinyl moiety has been substituted for the (R)-3-aminopiperidinyl moiety are also encompassed by the methods, compositions and techniques described herein.

Additional DPP-IV inhibitors that can be synthesized using variations of the methods described herein can be found in U.S. Patent Application Publication Nos. 2005/0004117, 2004/0259870, 2004/0242568, and 2004/0242566, the disclosures of which are incorporated by reference in their entirety.

Synthesis of Compounds of (R)-3-aminopiperidine

The methods described herein minimize the need for chiral purification techniques and yield at least 95%, optionally at least 97%, optionally at least 98% and optionally at least 99% enantiomerically pure (R)-3-aminopiperidine dihydrochloride. Further, the methods described herein utilize readily available reagents and techniques that may be scaled to produce either small or large quantities of (R)-3-aminopiperidine dihydrochloride. Further, the methods described herein may be used to produce at least about 1 kilogram of (R)-3-aminopiperidine dihydrochloride, at least about 2 kilograms of (R)-3-aminopiperidine dihydrochloride, at least about 4 kilograms of (R)-3-aminopiperidine dihydrochloride, at least about 10 kilograms of (R)-3-aminopiperidine dihydrochloride, or at least about 100 kilograms of (R)-3-aminopiperidine dihydrochloride. Further, the methods described herein are reproducible, reliable and amenable to the manufacture of (R)-3-aminopiperidine dihydrochloride under the good manufacturing practices necessary for regulatory approval of drugs and drug products.

If needed to further improve the enantiomeric excess of the 3-aminopiperidines described herein, various methods for resolving and/or separating mixtures of different stereoisomers known in the art may be used. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) that they can be readily separated by taking advantage of these dissimilarities. For example, diastereomers can typically be separated by chiral chromatography or by separation/resolution techniques based upon differences in solubility. The optically pure enantiomer is then recovered, along with the resolving agent, by any practical means that would not result in racemization. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981), which is hereby incorporated by reference in its entirety.

An exemplary method for synthesizing a chiral 3-aminopiperidine is presented in Scheme 1 below, using, by way of example, the synthesis of (R)-3-aminopiperidine.

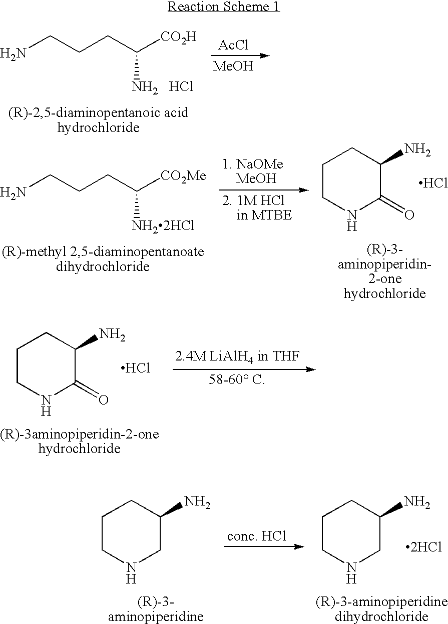

The first step in reaction Scheme 1 involves the esterification of (R)-2,5-diaminopentanoic acid hydrochloride (also known as D-omithine) by the conversion of the amino acid to an amino acyl chloride by use of a reagent capable of such a conversion. In one embodiment, acetyl chloride is used for the transformation, however, other suitable reagents may be used. Following this conversion, an alcohol reacts with the amino acyl chloride to form the desired ester. Various alcohols may be used in this step to give the corresponding esters. Such alcohols include, but are not limited to, methanol, ethanol, propanol, isopropanol, butanol, pentanol, and hexanol. In the example, the esterification step involves mixing the (R)-2,5-diaminopentanoic acid hydrochloride and an alcohol together and cooling the mixture to below RT, by way of example at temperatures between about 0° C. and about 15° C., followed by addition of about 1.5 to about 2.5 equivalents of acetyl chloride at a temperature below RT, by way of example at temperatures between about 0° C. and about 15° C. The resulting mixture is then heated, by way of example at temperatures between about 45° C. and about 65° C., and the resulting ester may be isolated and purified or it may be used in subsequent synthetic steps without isolation or purification. A non-limiting approach to this esterification step is to dissolve (R)-2,5-diaminopentanoic acid hydrochloride in methanol and cool the mixture to between about 5° C. and about 10° C. About 2.0 equivalents of acetyl chloride are then controllably added while maintaining the temperature between about 5° C. and about 10° C. Upon completion of acetyl chloride addition, the mixture is then heated to between about 50° C. and 60° C. until the reaction is complete, or near completion, as determined by measurement techniques, by way of example, ¹H NMR spectrometry. The resulting methyl ester, (R)-methyl 2,5-diaminopentanoate dihydrochloride (also known as D-omithine methyl ester dihydrochloride), may then be isolated and purified or it may be used in subsequent synthetic steps without isolation or purification. Such esterification methods can be used with derivatives of 2,5-diaminopentanoic acid hydrochloride, the (S)-enantiomer of 2,5-diaminopentanoic acid hydrochloride and its derivatives, with acid addition salts other than HCl, and with protected versions of such compounds.

The ester formed in the first step of reaction Scheme 1 is then used in the formation of (R)-3-aminopiperidin-2-one hydrochloride as shown in step 2 of reaction Scheme 1. In step 2 of reaction Scheme 1 (R)-3-aminopiperidin-2-one hydrochloride is formed by the cyclization of the ester formed in the first step of reaction Scheme 1, followed by salt formation. Cyclization results in the formation of (R)-3-aminopiperidin-2-one which is formed by dissolving the ester in an alcohol, cooling the mixture to temperatures below about 0° C., by way of example at temperatures between about −10° C. and about 0° C., and then slowly adding about 1.5 to 3.0 equivalents a metal alkoxide at temperatures below about 0° C., by way of example at temperatures between about −10° C. and about 0° C. Alternatively, any similar, non-resin based source of anions may be used.

Following cyclization, about 1.0 to about 1.5 equivalents of an acid in an organic solvent are added to form the corresponding (R)-3-aminopiperidin-2-one salt at temperatures between about 0° C. and about 20° C. The acid in organic solvent may be formed by adding an acid to an organic solvent, or the acid may be formed in situ. For example, a 1 M solution of HCl in MTBE may be formed by adding acetyl chloride to a solution of MeOH in MTBE. The organic solvent used is such that the solubility of the resulting salt is low, allowing precipitation to readily occur followed by isolation by filtration. Appropriate salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartatic acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

A non-limiting approach to step 2 of reaction Scheme 1 is to dissolve (R)-methyl 2,5-diaminopentanoate dihydrochloride in methanol and cool the solution to between about −10° C. and about −5° C., followed by controlled addition of about 2.6 equivalents of sodium methoxide at temperatures between about −10° C. and about −5° C. This reaction mixture is then stirred, while maintaining the temperatures between about −10° C. and about −5° C., until the reaction is complete, or near complete, as determined by measurement techniques such as, by way of example, ¹H NMR spectrometry. Upon completion, the resulting (R)-3-aminopiperidin-2-one hydrochloride may be obtained by addition of a 1 M solution of HCl in MTBE while maintaining the temperatures between about 5° C. and about 15° C. The HCl addition salt precipitates from solution and may be isolated by simple filtration. The HCl addition salt may be used to further produce (R)-3-aminopiperidine hydrochloride. Such cyclization and acid addition salt formation methods can be used with other esters of 2,5-diaminopentanoic acid, other acid addition salts of such esters, the (S)-enantiomer of such esters and acid addition salts, other derivatives of such esters and acid addition salts, and with protected versions thereof.

The conversion of (R)-3-aminopiperidin-2-one into a salt prior to use in subsequent reaction steps simplifies the synthesis of (R)-3-aminopiperidine hydrochloride. For example, use of such a salt eliminates the need to isolate and purify (R)-3-aminopiperidin-2-one, which requires a distillation step and special equipment compared to simple filtration as described herein. By way of example, such distillation processes may involve vacuum distillation and may have further complications relative to simple filtration. In contrast to using a filtration system, as described herein, vacuum distillation requires maintenance of a correct vacuum and correct temperature to achieve effective distillation.

The methods described herein may be used to produce multi-kilogram quantities of (R)-3-aminopiperidine hydrochloride in a single series of contiguous reactions. In this regard, the methods described herein avoid use of an anion exchange resin, which is limited to producing small amounts of (R)-3-aminopiperidin-2-one (less than 10 grams). Further, the procedures described herein, avoid the need for regeneration (and/or disposal) of a resin.

In the third step of reaction Scheme 1, the (R)-3-aminopiperidin-2-one hydrochloride is reduced by a reducing agent to form (R)-3-aminopiperidine. About 1.0 to about 2.5 equivalents of the reducing agent is slowly added to (R)-3-aminopiperidin-2-one hydrochloride in a solvent at a temperature between about 10° C. and about 45° C. Upon completion of the addition of the reducing agent, the resulting mixture is heated at a temperature between about 45° C. and 70° C. A non-limiting approach to step 3 of reaction Scheme 1 is to dissolve (R)-3-aminopiperidin-2-one hydrochloride in THF and cool the mixture to between about 25° C. and about 35° C., followed by controlled addition of about 1.6 equivalents of lithium aluminum hydride in THF. This reaction mixture is then heated, at temperatures between about 55° C. and about 65° C., until the reaction is complete, or near complete, as determined by a measurement technique, such as TLC analysis.

The resulting (R)-3-aminopiperidine is subsequently treated with HCl (fourth step of reaction Scheme 1) to obtain (R)-3-aminopiperidine dihydrochloride. Other salts that can be formed include acid addition salts formed with inorganic acids such as hydrobromic acid, sulfuric acid, nitric acid, phosphoric acid, and the like; and organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartatic acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. The (R)-3-aminopiperidine hydrochloride may be isolated by filtration for later use in the synthesis of DPP-IV inhibitors.

Such reduction and acid addition salt formation methods can be used with other derivatives of 3-aminopiperidin-2-one, other acid addition salts of 3-aminopiperidin-2-one and such derivatives, the (S)-enantiomer of 3-aminopiperidin-2-one and such derivatives and acid addition salts, as well as with protected versions thereof.

The synthetic methods used to obtain (R)-3-aminopiperidine hydrochloride, as described herein, have certain beneficial characteristics. First, the generation and utilization of the salt forms of the products of steps 2 and 4 significantly simplifies the synthetic process by allowing product isolation and purification using simple filtration techniques, thus avoiding the need to purify products by more complex procedures, including distillation methods requiring special equipment. Second, the synthetic methods described herein can be used to obtain large quantities of (R)-3-aminopiperidine hydrochloride from readily available D-ornithine in a cost effective manner. Third, the synthetic methods described herein are solution based and do not depend on surface-based reactions, such as ion exchange resins. For surface-chemistry-driven reactions, the condition/reactivity of the surface needs to be reproducibly generated/regenerated for each use to ensure reproducible synthesis of a desired product. In addition, surface-based reactions become impracticable for producing kilogram quantities of product because of expense, storage, and disposal concerns. Further, the synthetic methods disclosed herein do not rely on the reproducibility of a surface and therefore the quantities of products are reproducibly synthesized. Fourth, the synthetic methods described herein retain the stereochemistry initially present in the starting material. Thus, the (R)-3-aminopiperidine hydrochloride can be used directly in the synthesis of various DPP-IV inhibitors without the need to further purify or resolve mixtures of 3-aminopiperidine hydrochloride of lower enantiomeric purity.

### Examples

The following examples provide illustrative methods for the synthesis of (R)-3-aminopiperidine hydrochloride and the various intermediate compounds described in reaction scheme 1. These examples are provided for illustrative purposes only and not to limit the scope of the claims provided herein. Further, as stated throughout, the following methods may be used with other enantiomers, derivatives, acid addition salts, and esters of such compounds, as well as with protected forms thereof.

### Example 1

### Synthesis of D-Ornithine methyl ester hydrochloride ((R)-methyl 2,5-diaminopentanoate dihydrochloride)

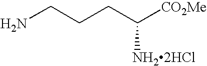

A clean and dry jacketed reactor, equipped with a reflux condenser, a temperature probe and an addition funnel or head column, was flushed with nitrogen gas for 15 minutes prior to the addition of 5000 g of D-ornithine hydrochloride and 25 L of methanol into the reactor. The reactor was then cooled to 5-10° C. Once the batch was at 5-10° C., 4.2 L of acetyl chloride (4700 g, 60 mol) was slowly added over 3 hours using an addition funnel while allowing the reactor temperature to rise to, and then be maintained at, between 5 to 20° C. Once the addition was completed, the batch was heated to 50-60° C. and stirred at that temperature for 3 hours, during which the slurry became a homogeneous solution. Progress of the reaction was monitored using ¹H NMR spectrometry until the reaction appeared complete. Once the reaction was complete, the mixture was cooled to between −10 to −5° C. in preparation for the synthesis of (R)-3-aminopiperidin-2-one hydrochloride. The product (R)-methyl 2,5-diaminopentanoate dihydrochloride was not isolated and remained in the reactor.

### Example 2

### Synthesis of (R)-3-aminopiperidin-2-one hydrochloride

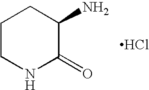

Without isolating the (R)-methyl 2,5-diaminopentanoate dihydrochloride synthesized in Example 1, the synthesis of (R)-3-aminopiperidin-2-one hydrochloride was continued using the same reactor as in Example 1. The solution containing (R)-methyl 2,5-diaminopentanoate dihydrochloride was cooled to between −10 to −5° C. and 16,571 g of sodium methoxide (25 wt % in MeOH, 15 L, 66 mol)) was added over 3 hours using an addition funnel while maintaining the temperature between −10-0° C. during the addition. The pH of the reaction mixture was checked throughout the addition to ensure the pH did not exceed 10. During the addition of sodium methoxide, a thick white slurry was formed. After the addition was complete, the mixture was stirred at a temperature between −10 to 0° C. for 1.5 hours, and the progress of the reaction monitored using ¹H NMR spectrometry until the reaction appeared complete. When the reaction appeared complete, 40 L of MTBE was added to the mixture while maintaining the temperature between 10 to 25° C. After 30 minutes, the mixture was filtered over Celite using a Hastelloy Nütsche filter and the filter was washed with a 1:1 mixture of 5 L of MTBE and 5 L of methanol. The resulting filtrate was concentrated under reduced pressure to 20% of the original filtrate volume using a rotary evaporator, during which the temperature of the rotavap bath was maintained at about 30° C. The resulting solution containing (R)-3-aminopiperidin-2-one was transferred into a clean, dry reactor and cooled to between 5 to 10° C. in preparation for synthesis of the salt (R)-3-aminopiperidin-2-one hydrochloride (see below).

### Preparation of 1M solution of HCl in MTBE and Reaction with (R)-3-aminopiperidin-2-one

A second reactor was filled with 30 L of MTBE and 3 L of methanol, and then 2.3 L of acetyl chloride (2600 g, 33 mol) was added over 2 hours while the temperature was maintained at 15 to 25° C. The reactor containing the solution of (R)-3-aminopiperidin-2-one was cooled to a temperature between to 5-10° C. and the pH of the solution was adjusted to between 4-5 by slowly adding the hydrogen chloride solution from the reactor containing the MTBE, methanol, and acetyl chloride. The temperature was maintained between 5 to 15° C. and the pH was monitored throughout the addition of the HCl solution. 38 L of MTBE was then added to the mixture while the temperature was maintained between 5 to 15° C. and the mixture was stirred for 60 minutes. The resulting salt precipitated from solution and created a slurry which was stirred for 30-60 minutes at 10 to 25° C. Afterwards, the resulting salt was filtered using a Hastelloy Nütsche filter and washed with 13 L of MTBE. The resulting (R)-3-aminopiperidin-2- one hydrochloride was then dried to a constant weight in a vacuum oven at 20-25° C. and then analyzed using ¹H NMR (DMSO-d₆). The chiral purity of the product was determined by derivatization with Mosher's acid chloride.

### Example 3

### Synthesis of (R)-3-aminopiperidine

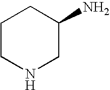

4000 g of the (R)-3-aminopiperidin-2-one hydrochloride obtained in Example 2 was added to a clean and dry jacketed reactor which had been flushed with nitrogen gas for 15 minutes. The reactor was also equipped with a syltherm reflux condenser, a temperature probe and an addition funnel. Also, a Claisen adaptor was attached to the top of the condenser and a nitrogen inlet was attached to one of the ports and a vent attached to the other port. 60 L of THF was then added to the reactor containing the (R)-3-aminopiperidin-2-one hydrochloride, during which time the reactor was continually flushed with nitrogen gas. Then, 17 L of 2.4M lithium aluminum hydride (LiAH) solution in THF was added dropwise over 2.2 hours using the addition funnel. During this addition step, the temperature was maintained below 35° C. After the addition was complete, the resulting heterogeneous mixture was heated to 58-60° C. and stirred for 1.2 hours. After 1 hour at this temperature, the progress of the reaction was assayed using TLC (50% MeOH/CH₂Cl₂ or 60% MeOH:DCM, I₂ stain or ninhydrin stain) until the reaction appeared complete. After completion of the reaction, the mixture was cooled to 20-25° C. overnight and then between 0-5° C. Meanwhile, 12 L of DI water was added to a clean addition funnel which was then carefully placed onto the reactor. The reactor was flushed with nitrogen prior to the addition of the DI water, and a constant nitrogen atmosphere was maintained during addition. The mixture was stirred while the DI water was added dropwise at such a rate that the temperature of the batch was maintained below 50° C. Once the water addition was complete, 20 L of methanol was added to the mixture over 10 minutes using an addition funnel and the batch strirred for 45 minutes while the temperature was maintained between 45-50° C. The mixture was then cooled to between 35-45° C., and the resulting slurry was filtered over Celite using a clean filter (glass or Nütsche) and then washed twice with 15 L of THF (40 L total). The combined filtrates containing the product (R)-3-aminopiperidine were then transferred back into the reactor, and the mixture was cooled to between 0-10° C., in preparation for the salt formation step (Example 4).

### Example 4

### Synthesis of (R)-3-aminopiperidine dihydrochloride

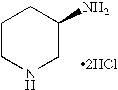

The mixture containing the (R)-3-aminopiperidine synthesized in Example 3 was transferred back into the reactor, and the mixture was cooled to between 0-10° C. in preparation for the synthesis of (R)-3-aminopiperidine dihydrochloride. Once this temperature was reached, 5.2 L of concentrated HCl was slowly added via an addition funnel, while the temperature was maintained between 15-25° C. The biphasic mixture was concentrated and the residual water azeotropically removed with reagent alcohol (2×36 L) and diluted in a minimal amount of alcohol (4-5 L). The residual THF content was monitored by ¹H NMR until an acceptable level (<5%) was reached. When the amount of THF remaining in the ethanol solution was less than 5 % as determined by ¹H NMR, the ethanol solution was transferred back to the jacketed reactor and cooled to between 25-30° C. After the temperature of the solution was between 25-30° C., 30 L of acetonitrile was slowly added to the reactor while the mixture was stirred at a temperature below 30° C. The resulting mixture was cooled to between 0-5° C. and stirred for 1 hour. The (R)-3-aminopiperidine dihydrochloride precipitated from solution during addition of acetonitrile. The resulting slurry was then filtered over a Hastelloy filter and the resulting (R)-3-aminopiperidine dihydrochloride solid was dried under vacuum at 20-35° C. and analyzed using ¹H, ¹³C NMR (DMSO-d₆), Chiral HPLC, and Karl Fisher analysis.

The examples and embodiments described herein are for illustrative purposes only and various modifications or changes suggested to persons skilled in the art are to be included within the spirit and purview of this application and scope of the appended claims. All publications, patents, and patent applications cited herein are hereby incorporated by reference for all purposes.

1. A method for making (R)-3-aminopiperidine dihydrochloride comprising:
forming a reaction mixture comprising (R)-3-aminopiperidin-2-one hydrochloride and between 1.0 and 2.5 equivalents of lithium aluminum hydride (relative to (R)-3-aminopiperidin-2-one hydrochloride) in a solvent comprising tetrahydrofuran;
maintaining the reaction mixture at a temperature between 45° C. and 70° C. for a time sufficient to form (R)-3-aminopiperidine; and
reacting the (R)-3-aminopiperidine with hydrochloric acid under conditions sufficient to form (R)-3-aminopiperidine dihydrochloride with at least 95% enantiomeric purity.

2. The method of claim 1, wherein the solvent in the reaction mixture is tetrahydrofuran.

3. The method of claim 1, wherein the reaction mixture is formed with between 1.5 and 2.0 equivalents of lithium aluminum hydride.

4. The method of claim 1, wherein the reaction mixture is formed with between 1.5 and 1.7 equivalents of lithium aluminum hydride.

5. The method of claim 1, wherein the reaction mixture is formed with 1.6 equivalents of lithium aluminum hydride.

6. The method of claim 1, wherein the reaction mixture is formed with at least one kilogram of (R)-3-aminopiperidin-2-one hydrochloride.

7. The method of claim 1, wherein the reaction mixture is formed with at least two kilograms of (R)-3-aminopiperidin-2-one hydrochloride.

8. The method of claim 1, wherein the (R)-3-aminopiperidine dihydrochloride formed has a chiral purity of at least 95%.

9. The method of claim 1, wherein the (R)-3-aminopiperidine dihydrochloride formed has a chiral purity of at least 97%.

10. The method of claim 1, wherein the (R)-3-aminopiperidine dihydrochloride formed has a chiral purity of at least 98%.

11. The method of claim 1, wherein the (R)-3-aminopiperidine dihydrochloride formed has a chiral purity of at least 99%.

12. The method of claim 1, wherein the reaction mixture is formed with at least one kilogram of (R)-3-aminopiperidin-2-one hydrochloride.

13. The method of claim 1, wherein the reaction mixture is formed with at least two kilograms of (R)-3-aminopiperidin-2-one hydrochloride.

14. The method of claim 1, wherein the reaction mixture is formed with at least four kilograms of (R)-3-aminopiperidin-2-one hydrochloride.

15. The method of claim 1, wherein the reaction mixture is formed with at least ten kilograms of (R)-3-aminopiperidin-2-one hydrochloride.

16. The method of claim 1, wherein the reaction mixture is formed with at least one hundred kilograms of (R)-3-aminopiperidin-2-one hydrochloride.

17. The method of claim 1, wherein the reaction mixture is formed at a temperature between 15° C. and 35° C.

18. The method of claim 1, wherein the reaction mixture is formed at a temperature between 25° C. and 35° C.

19. The method of claim 1, wherein the reaction mixture is maintained at a temperature between 55° C. and 65° C. when forming (R)-3-aminopiperidine.

20. The method of claim 1, wherein the (R)-3-aminopiperidine dihydrochloride formed is isolated by a method comprising filtration.

21. The method of claim 1, wherein the (R)-3-aminopiperidin-2-one hydrochloride is formed by a method comprising
forming a mixture comprising (R)-methyl-2,5-diaminopentanoate dihydrochloride and between 1.5 and 3 equivalents of sodium methoxide (relative to (R)-methyl-2,5-diaminopentanoate dihydrochloride) in a solvent comprising methanol;
maintaining the mixture at a temperature between −10° C. and 0° C. for a time sufficient to form (R)-3-aminopiperidin-2-one;
forming a mixture comprising (R)-3-aminopiperidin-2-one and between 1.0 and 3.0 equivalents of hydrochloric acid (relative to (R)-3-aminopiperidin-2-one) in a solvent comprising methanol; and
maintaining the mixture at a temperature between 0° C. and 20° C. for a time sufficient to form (R)-3-aminopiperidin-2-one hydrochloride.

22. The method of claim 19, wherein the solvent in which the mixture comprising (R)-methyl-2,5-diaminopentanoate dihydrochloride is formed comprises methyl tert-butyl ether and methanol.

23. The method of claim 19, wherein between 2.5 and 2.7 equivalents of sodium methoxide is used to form the mixture.

24. The method of claim 19, wherein 2.6 equivalents of sodium methoxide is used to form the mixture.

25. The method of claim 19, wherein forming a mixture comprising (R)-methyl-2,5-diaminopentanoate dihydrochloride is performed at a temperature between -10° C. and −5° C.

26. The method of claim 19, wherein forming the (R)-3-aminopiperidin-2-one is performed at a temperature between −10° C. and −5° C.

27. The method of claim 19, wherein reacting the (R)-3-aminopiperidin-2-one with hydrochloric acid is performed at a temperature between 5° C. and 15° C.

28. The method of claim 19, wherein the (R)-3-aminopiperidin-2-one hydrochloride is isolated by a method comprising filtration.

29. The method of claim 19, wherein the (R)-methyl-2,5-diaminopentanoate dihydrochloride is synthesized by a method comprising
forming a mixture comprising (R)-2,5-diaminopentanoic acid hydrochloride and between 1.5 and 2.5 equivalents of acetyl chloride (relative to (R)-2,5-diaminopentanoic acid hydrochloride) in a solvent comprising methanol; and
maintaining the mixture at a temperature between 45° C. and 65° C. for a time sufficient to form (R)-methyl-2,5-diaminopentanoate dihydrochloride.

30. The method of claim 27, wherein the solvent in which the mixture comprising (R)-2,5-diaminopentanoic acid hydrochloride is formed is methanol.

31. The method of claim 27, wherein between 1.9 and 2.1 equivalents of acetyl chloride is used to form the mixture comprising (R)-2,5-diaminopentanoic acid hydrochloride.

32. The method of claim 27, wherein 2 equivalents of acetyl chloride is used to form the mixture comprising (R)-2,5-diaminopentanoic acid hydrochloride.

33. The method of claim 27, wherein the mixture comprising (R)-2,5-diaminopentanoic acid hydrochloride comprises at least 1 kilogram of (R)-2,5-diaminopentanoic acid hydrochloride.

34. The method of claim 27, wherein forming a mixture comprising (R)-2,5-diaminopentanoic acid hydrochloride is performed at a temperature between 0° C. and about 15° C.

35. The method of claim 27, wherein forming a mixture comprising (R)-2,5-diaminopentanoic acid hydrochloride is performed at a temperature between 5° C. and about 10° C.

36. The method of claim 27, wherein form (R)-methyl-2,5-diaminopentanoate dihydrochloride is performed at a temperature between 50° C. and about 60° C.

37. The method of claim 19, wherein the (R)-3-aminopiperidin-2-one hydrochloride formed has a chiral purity of at least 95%.

38. The method of claim 19, wherein the (R)-3-aminopiperidin-2-one hydrochloride formed has a chiral purity of at least 97%.

39. The method of claim 19, wherein the (R)-3-aminopiperidin-2-one hydrochloride formed has a chiral purity of at least 98%.

40. The method of claim 19, wherein the (R)-3-aminopiperidin-2-one hydrochloride formed has a chiral purity of at least 99%.

41. The method of claim 19, wherein the reaction mixture is formed with at least one kilogram of (R)-methyl-2,5-diaminopentanoate dihydrochloride.

42. The method of claim 19, wherein the reaction mixture is formed with at least two kilograms of (R)-methyl-2,5-diaminopentanoate dihydrochloride.

43. The method of claim 19, wherein the reaction mixture is formed with at least four kilograms of (R)-methyl-2,5-diaminopentanoate dihydrochloride.

44. The method of claim 19, wherein the reaction mixture is formed with at least ten kilograms of (R)-methyl-2,5-diaminopentanoate dihydrochloride.

45. The method of claim 19, wherein the reaction mixture is formed with at least one hundred kilograms of (R)-methyl-2,5-diaminopentanoate dihydrochloride.

46. A process of making (R)-3-aminopiperidine dihydrochloride comprising:
forming (R)-methyl 2,5-diaminopentanoate dihydrochloride by an esterification reaction of (R)-2,5-diaminopentanoic acid hydrochloride with methanol and acetyl chloride,
forming (R)-3-aminopiperidin-2-one hydrochloride by a cyclization reaction of(R)-methyl 2,5-diaminopentanoate dihydrochloride with a metal alkoxide in an alcohol, and a reaction with hydrochloric acid in methyl tert-butyl ether,
reducing (R)-3-aminopiperidin-2-one hydrochloride with lithium aluminum in THF at a temperature of at least 35° C., and
forming (R)-3-aminopiperidine dihydrochloride by a reaction of (R)-3-aminopiperidine with hydrochloric acid.

47. The method of claim 46, wherein the (R)-3-aminopiperidine dihydrochloride formed has a chiral purity of at least 95%.

48. The method of claim 46, wherein the (R)-3-aminopiperidine dihydrochloride formed has a chiral purity of at least 97%.

49. The method of claim 46, wherein the (R)-3-aminopiperidine dihydrochloride formed has a chiral purity of at least 98%.

50. The method of claim 46, wherein the (R)-3-aminopiperidine dihydrochloride formed has a chiral purity of at least 99%.

51. The method of claim 46, wherein at least one kilogram of (R)-3-aminopiperidine dihydrochloride is formed.

52. The method of claim 46, wherein at least two kilograms of (R)-3-aminopiperidine dihydrochloride is formed.

53. The method of claim 46, wherein at least four kilograms of (R)-3-aminopiperidine dihydrochloride is formed.

54. The method of claim 46, wherein at least ten kilograms of (R)-3-aminopiperidine dihydrochloride is formed.

55. The method of claim 46, wherein at least one hundred kilograms of (R)-3-aminopiperidine dihydrochloride is formed.

56. The method of claim 1, further comprising reacting (R)-3-aminopiperidine dihydrochloride with a pyrimidine derivative.

57. The method of claim 56, wherein the pyrimidine derivative comprises a compound of the formula: 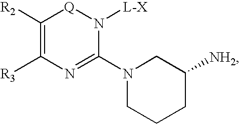
wherein:
Q is selected from the group consisting of CO, SO, SO₂, and C═NR₄;
Z′ is a leaving group;
R₂ and R₃ are each independently selected from the group consisting of hydrogen, halo, perhalo(C₁₋₁₀)alkyl, amino, cyano, nitro, thio, (C₁₋₁₀)alkyl, alkene, alkyne, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl group, imine group, sulfonyl group and sulfinyl group, each substituted or unsubstituted;
R₄ is selected from the group consisting of hydrogen, (C₁₋₁₀)alkyl, cycloalkyl, heterocycloalkyl, aryl, heteroaryl, arylalkyl, heteroarylalkyl, bicycloaryl, heterobicycloaryl, each substituted or unsubstituted;
L is a linker providing 0-6 atom separation between X and the ring to which L is attached; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

58. The method of claim 57, wherein Z′ is halo.

59. The method of claim 56, wherein reacting (R)-3-aminopiperidine dihydrochloride with the pyrimidine derivative forms a compound comprising the formula: 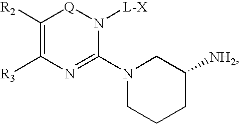
wherein:
Q is selected from the group consisting of CO, SO, SO₂, and C═NR₄;
R₂ and R₃ are each independently selected from the group consisting of hydrogen, halo, perhalo(C₁₋₁₀)alkyl, amino, cyano, nitro, thio, (C₁₋₁₀)alkyl, alkene, alkyne, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl group, imine group, sulfonyl group and sulfinyl group, each substituted or unsubstituted;
R₄ is selected from the group consisting of hydrogen, (C₁₋₁₀)alkyl, cycloalkyl, heterocycloalkyl, aryl, heteroaryl, arylalkyl, heteroarylalkyl, bicycloaryl, heterobicycloaryl, each substituted or unsubstituted;
L is a linker providing 0-6 atom separation between X and the ring to which L is attached; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

60. The method of claim 56, wherein reacting (R)-3-aminopiperidine dihydrochloride with the pyrimidine derivative forms a compound comprising a formula selected from the group consisting of

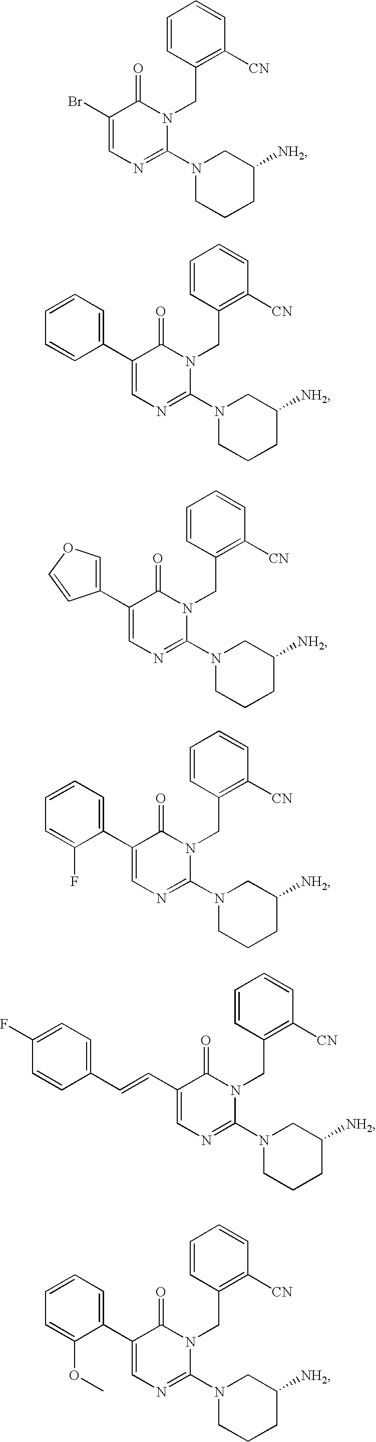

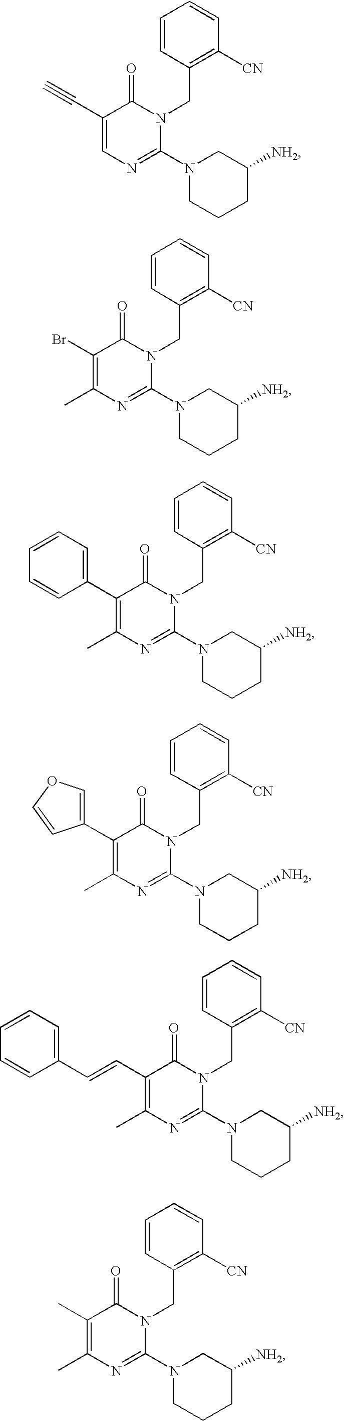

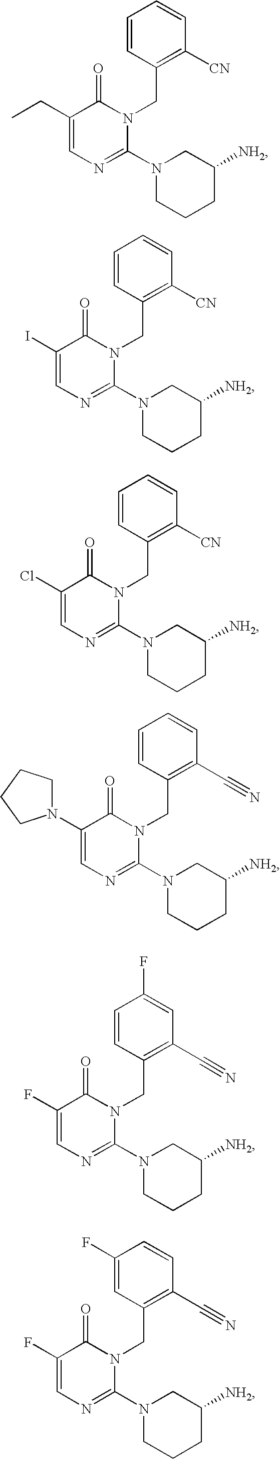

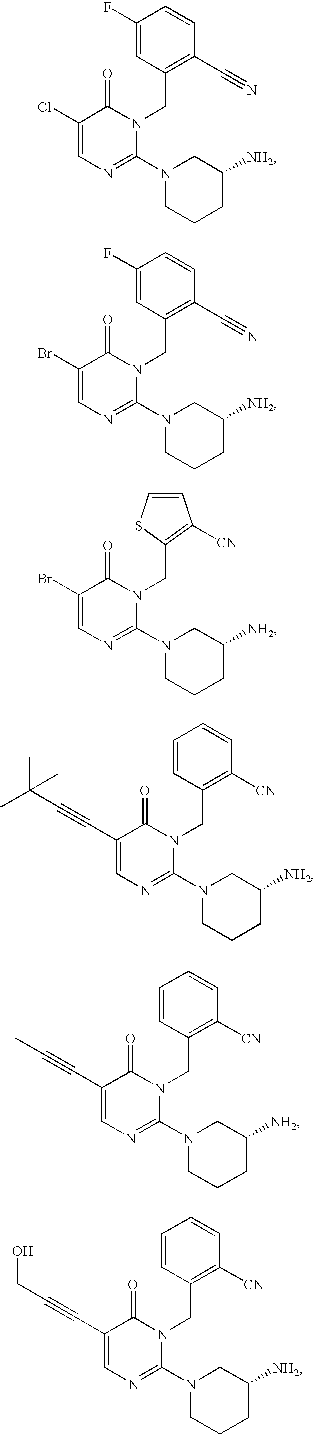

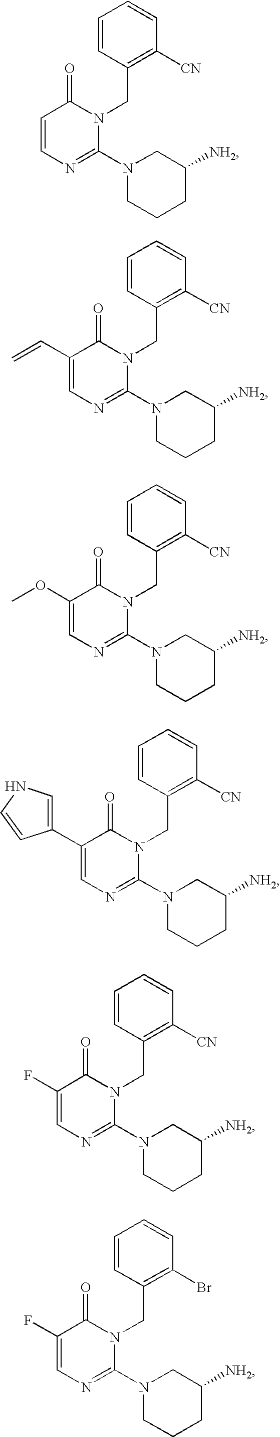

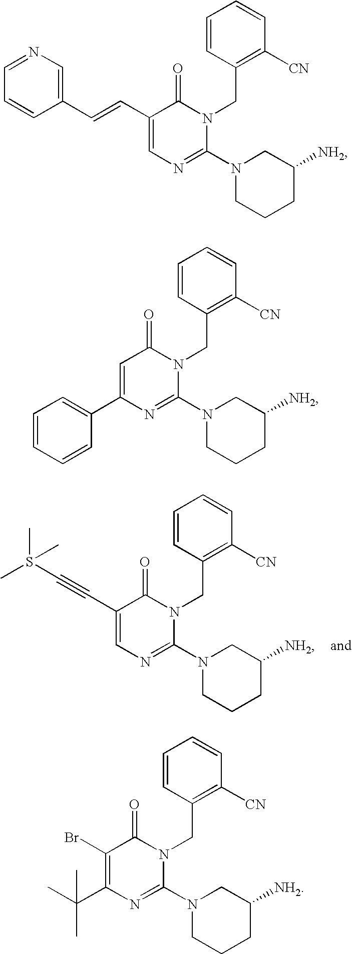

61. The method of claim 56, wherein reacting (R)-3-aminopiperidine dihydrochloride with the pyrimidine derivative forms a compound comprising the formula: 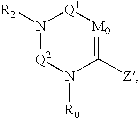
wherein:
M₀ is -C-LX, N or CR₄;
Z′ is a leaving group;
Q¹ and Q² are each independently selected from the group consisting of CO, CS, SO, SO₂, and C═NR₉;
R₀ is R₁ or -LX, with the proviso that only one of R₀ and M₀ is -LX;
R₁ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₂ is hydrogen or selected from the group consisting of (C₁₋₁₀)alkyl, (C₃₋₁₂)cycloalkyl, (C₃₋₁₂)cycloalkyl(C₁₋₅)alkyl, hetero(C₃₋₁₂)cycloalkyl(C₁₋₅)alkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, hetero(C₄₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl group, imino group, sulfonyl group and sulfinyl group, each substituted or unsubstituted;
R₄ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₉ is hydrogen or is selected from the group consisting of alkyl, cycloalkyl, heterocycloalkyl, arylalkyl, heteroarylalkyl, bicycloaryl, and heterobicycloaryl, each substituted or unsubstituted;
L is a linker providing 1, 2 or 3 atom separation between X and the ring to which L is attached, wherein the atoms of the linker providing the separation are selected from the group consisting of carbon, oxygen, nitrogen, and sulfur; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

62. The method of claim 61, wherein Z′ is halo.

63. The method of claim 56, wherein the reaction of the (R)-3-aminopiperidine dihydrochloride with the pyrimidine derivative provides a product comprising a compound of the formula 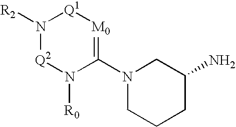
wherein:
M₀ is -C-LX, N or CR₄;
Q¹ and Q² are each independently selected from the group consisting of CO, CS, SO, SO₂, and C═NR₉;
R₀ is R₁ or -LX, with the proviso that only one of R₀ and M₀ is -LX;
R₁ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₂ is hydrogen or selected from the group consisting of (C₁₋₁₀)alkyl, (C₃₋₁₂)cycloalkyl, (C₃₋₁₂)cycloalkyl(C₁₋₅)alkyl, hetero(C₃₋₁₂)cycloalkyl(C₁₋₅)alkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, hetero(C₄₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl group, imino group, sulfonyl group and sulfinyl group, each substituted or unsubstituted;
R₄ is hydrogen or is selected from the group consisting of halo, perhalo(C₁₋₁₀)alkyl, amino, cyano, thio, (C₁₋₁₀)alkyl, cycloalkyl, heterocycloalkyl, arylalkyl, heteroarylalkyl, aryl, heteroaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, hydroxy, alkoxy, aryloxy, heteroaryloxy, carbonyl group, imino group, sulfonyl group and sulfinyl group, each substituted or unsubstituted;
R₉ is hydrogen or is selected from the group consisting of alkyl, cycloalkyl, heterocycloalkyl, arylalkyl, heteroarylalkyl, bicycloaryl, and heterobicycloaryl, each substituted or unsubstituted;
L is a linker providing 1, 2 or 3 atom separation between X and the ring to which L is attached, wherein the atoms of the linker providing the separation are selected from the group consisting of carbon, oxygen, nitrogen, and sulfur; and
X is selected from the group consisting of (C₁₋₁₀)alkyl, (C₃₋₁₂)cycloalkyl, hetero(C₃₋₁₂)cycloalkyl, aryl(C₁₋₁₀)alkyl, heteroaryl(C₁₋₅)alkyl, (C₉₋₁₂)bicycloaryl, hetero(C₄₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, alkenyl, alkynyl, carbonyl group, cyano, imino group, sulfonyl group and sulfinyl group, each substituted or unsubstituted.

64. The method of claim 56, wherein the reaction of the (R)-3-aminopiperidine dihydrochloride with the pyrimidine derivative provides a product comprising a compound selected from the group consisting of 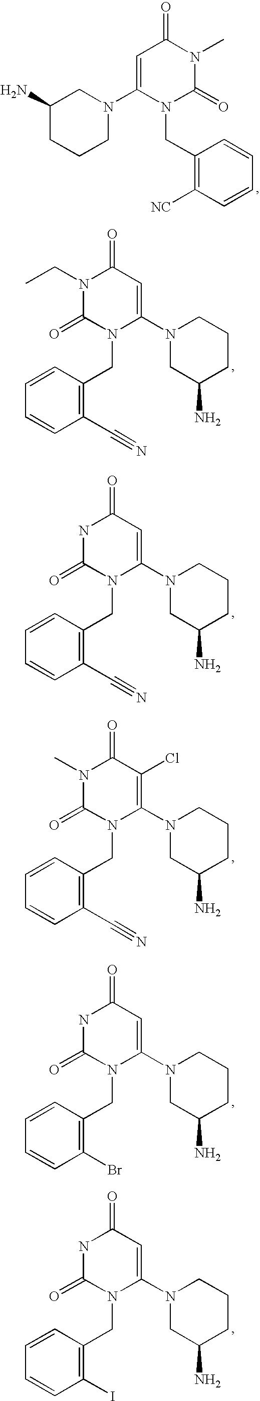 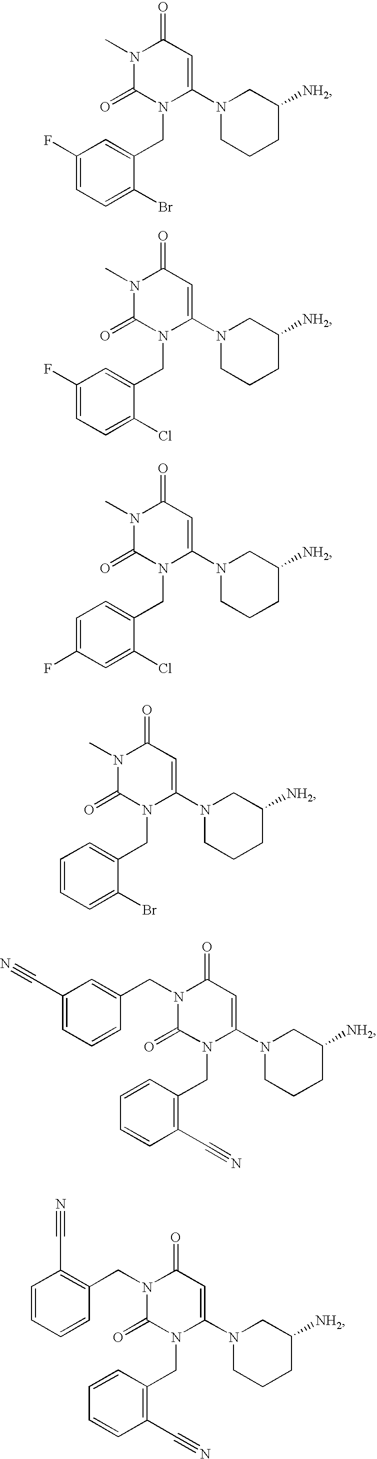 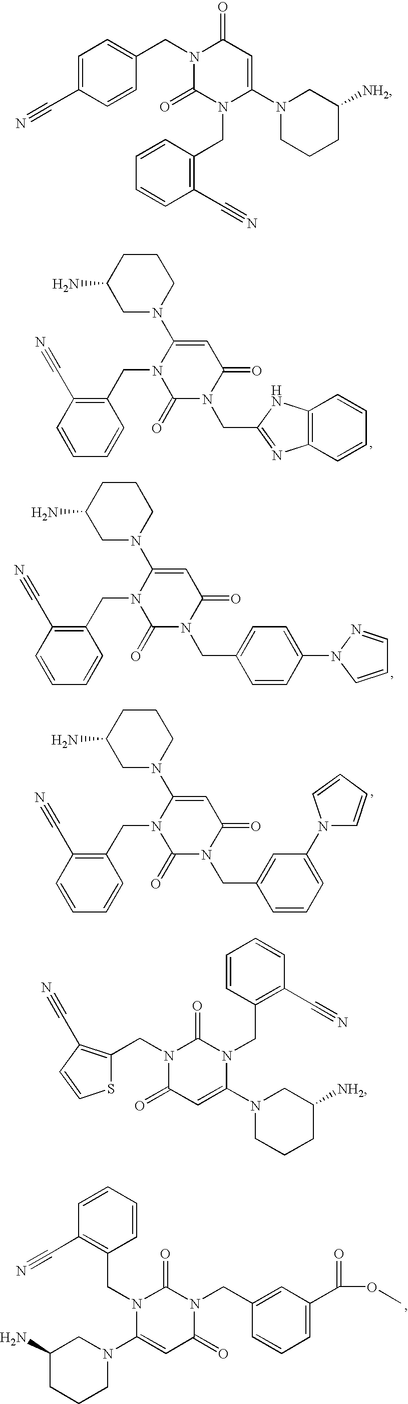 [chemical structure]
